SEQUENCE LISTING (22 sequences)

SEQ ID NO: 1 - protein, 5 aa, Artificial Sequence
  Description of Artificial Sequence Synthetic peptide
XKWAP

SEQ ID NO: 2 - protein, 5 aa, Artificial Sequence
  Description of Artificial Sequence Synthetic peptide
XKWAX

SEQ ID NO: 3 - protein, 6 aa, Artificial Sequence
  Description of Artificial Sequence Synthetic peptide
XSWPGP

SEQ ID NO: 4 - protein, 7 aa, Artificial Sequence
  Description of Artificial Sequence Synthetic peptide
XDGPIPP

SEQ ID NO: 5 - protein, 9 aa, Artificial Sequence
  Description of Artificial Sequence Synthetic peptide
XWPRPQIPP

SEQ ID NO: 6 - protein, 10 aa, Artificial Sequence
  Description of Artificial Sequence Synthetic peptide
XYWPGPNIPP

SEQ ID NO: 7 - protein, 10 aa, Artificial Sequence
  Description of Artificial Sequence Synthetic peptide
ENWPHPQIPP

SEQ ID NO: 8 - protein, 10 aa, Artificial Sequence
  Description of Artificial Sequence Synthetic peptide
XNWPHPQIPP

SEQ ID NO: 9 - protein, 10 aa, Artificial Sequence
  Description of Artificial Sequence Synthetic peptide
ENWPHPQIPP

SEQ ID NO: 10 - protein, 9 aa, Artificial Sequence
  Description of Artificial Sequence Synthetic peptide
NPWHPQIPP

SEQ ID NO: 11 - protein, 10 aa, Artificial Sequence
  Description of Artificial Sequence Synthetic peptide
XNWPHPQIPP

SEQ ID NO: 12 - protein, 9 aa, Artificial Sequence
  Description of Artificial Sequence Synthetic peptide
NWPHPQIPP

SEQ ID NO: 13 - protein, 11 aa, Artificial Sequence
  Description of Artificial Sequence Synthetic peptide
XARPPHPPIPP

SEQ ID NO: 14 - protein, 12 aa, Artificial Sequence
  Description of Artificial Sequence Synthetic peptide
XWGRPPGPPIPP

SEQ ID NO: 15 - protein, 13 aa, Artificial Sequence
  Description of Artificial Sequence Synthetic peptide
XGGWPRPGPEIPP

SEQ ID NO: 16 - protein, 7 aa, Artificial Sequence
  Description of Artificial Sequence Synthetic peptide
PHPQIPP

SEQ ID NO: 17 - protein, 10 aa, Artificial Sequence
  Description of Artificial Sequence Synthetic peptide
ARPPHPPIPP

SEQ ID NO: 18 - protein, 11 aa, Artificial Sequence
  Description of Artificial Sequence Synthetic peptide
WGRPPGPPIPP

SEQ ID NO: 19 - protein, 6 aa, Artificial Sequence
  Description of Artificial Sequence Synthetic peptide
WKYMVM

SEQ ID NO: 20 - protein, 8 aa, Artificial Sequence
  Description of Artificial Sequence Synthetic peptide
XWPRPQIP

SEQ ID NO: 21 - protein, 5 aa, Artificial Sequence
  Description of Artificial Sequence Synthetic peptide
XWPRP

SEQ ID NO: 22 - protein, 9 aa, Artificial Sequence
  Description of Artificial Sequence Synthetic peptide
XNWPHPQIP

(19) United States
(12) Patent Application Publication
(10) Pub. No.: US 2010/0035822 A1
(43) Pub. Date: Feb. 11, 2010

(54) PROLINE-RICH PEPTIDES, PHARMACEUTICAL COMPOSITION, USE OF ONE OR MORE PEPTIDES AND METHOD OF TREATMENT

(75) Inventors: Antônio Camargo, Sao Paulo (BR); Danielle Ianzer, Sao Paulo (BR); Carlos Silvia, Sao Paulo (BR); Claudiana Gomes, Sao Paulo (BR); Juliano Guerreiro, Sao Paulo (BR)

Correspondence Address: SMITH, GAMBRELL & RUSSELL, SUITE 3100, PROMENADE II, 1230 PEACHTREE STREET, N.E., ATLANTA, GA, 30309-3592, US

(73) Assignee: PEP FARMA CONSULTORIA E DESENVOLVIMENTO FARMACEUTI, Sao Paulo (BR)

(21) Appl. No.: 11/813,346

(22) PCT Filed: Jan. 5, 2007
(86) PCT No.: PCT/BR07/00003

§ 371 Date: Jul. 3, 2007

(30) Foreign Application Priority Data
Jan. 9, 2006 (BR) P10600331-1
Dec. 26, 2006 (BR) 20060190485

(51) Int. Cl. A61K 38/08; C07K 7/06

(52) U.S. Cl. 514/15; 530/330; 530/328; 530/327; 514/17

(57) ABSTRACT

The present invention provides oligopeptides with amino acid sequences comprised in set forth in SEQ ID NO 1 to 18, capable of binding to diverse targets, generating increase and sustenance to nitric oxide (NO) production in mammalian cells, potentiate the argininosuccinate synthase activity of animal cells and/or increase the intracellular bivalent calcium ion in animal cells. Also disclosed are pharmaceutical compositions of one or more peptides described in the present invention. The invention further relates to the use of these peptides.

### FIELD OF THE INVENTION

Peptides of the present invention are able to bind to diverse targets, promoting an increase and sustenance to the production of nitric oxide (NO) in mammalian cells. As such, peptides of the present invention act synergistically in the production of nitric oxide in cells by potentiating the activity of argininosuccinate synthase (AsS), and/or by increasing the intracellular concentration of calcium ion [Ca⁺²]ᵢ. Due to their ability to converge their activities to the sustained production of NO, these peptides are useful for the treatment and/or the prevention of pathologies involving NO deficiencies, including, among others, the cardiovascular diseases.

### BACKGROUND OF THE INVENTION

Recent studies have shown that reduction in nitric oxide (NO) concentration is related to several pathologies, particularly to cardiovascular dysfunctions such as arterial hypertension, coronary disease and congestive cardiac insufficiency.

Being an important paracrine mediator, NO is also crucial for the regulation of other essential systems beside the cardiovascular, such as the central and peripheral nervous systems, the excretion, the gastrointestinal and the immune systems, as well as, for the systemic, renal, and coronary haemodynamics. Thus, NO deficiency is directly related to several pathologies, comprising, beside cardiovascular diseases cited above, disorders of the nervous system, of the gastrointestinal system, of the immune system, neurodegenerative pathologies, pre-eclampsia, lymphocyte dysfunction during immune response and/or tumor growth, and, in addition, erectile dysfunction [Bredt D. S. Endogenous nitric oxide synthesis: biological functions and pathophysiology. Free Radical Res. 31: 577-596, 1999].

NO deficiency could be the result of an increase in the rate of NO inactivation, a reduction of NO synthesis, or a combination of both. A number of drugs and treatments aiming at increasing the amount of available and/or produced NO have been developed in order to treat and/or prevent cardiovascular diseases, as well as to treat other pathologies related to NO deficiency. NO produced by endothelial cells, for instance, rapidly diffuses to the vascular smooth muscle cells, and promotes vasodilation. The central or peripheral nervous tissue can also generate NO. In the nervous system NO functions as neurotransmitter, and exerts an important role in modulating the sympathetic activity, for instance, thus participating in the physiology and pathology of arterial blood pressure control [Ramchandra R, Barrett C J, Malpas S C. Nitric oxide and sympathetic nerve activity in the control of blood pressure. Clin Exp Pharmacol Physiol. 32(5-6):440-446, 2005. Review].

NO is synthesized from L-arginine, which is firstly converted into the intermediate N^{G}-hydroxy-L-arginine in the presence of nicotine-adenine-dinucleotide-phophate-hydrogen (NADPH) and the bivalent calcium ion (Ca²⁺). Subsequently, N^{G}-hydroxy-L-arginine is converted into L-citrulline and NO in the presence of NADPH and oxygen (O₂), and one of the three nitric oxide synthase isoenzymes, the hemeproteins NOS. To date three isoenzymes have been isolated, two of them being constitutively expressed (NOS-isoforms I and III), and one being induced (iNOS-isoform II) [Bredt D. S. Endogenous nitric oxide synthesis: biological functions and pathophysiology. Free Radical Res. 31: 577-596, 1999].

Another very important enzyme needed for the synthesis of NO is the argininosuccinate synthase (AsS), which catalyses the rate limiting step in citrulline recycling (produced during NO synthesis), into arginine (the substrate for NO synthesis), thereby promoting the sustained production of NO in endothelial cells [Pendleton et al., J. Biol. Chem., 280:24252-24260 (2005)]. The co-localization of AsS and endothelial NOS (eNOS) was demonstrated to be in the caveola, a compartment of the cytoplasm membrane of endothelial cells. In the absence of stimuli, recycling levels are minimal in these cells. However, once the NOS is stimulated, as for instance by the increase of intracellular concentration of Ca²⁺[Ca²⁺]ᵢ, over 80% of the produced citrulline is recycled into arginine by the citrulline-NO pathway, thereby activating the sustained production of NO. Thus, an efficient caveolar recycling complex supports the receptor mediated stimulation of endothelial NO production [Solomonson et al., J. Exp. Biol. 206:2083-2087 (2003); Shuttleworth, et al., Neurosci., 68:1295-1304 (1995)].

In the endothelium, NO functions as a vasoprotector, antagonizing contractions of the smooth muscles of the vessels, inhibiting the activation of the platelets, and acting upon integrins by modifying the leukocyte adhesion, and neutrophyle diapedesis [Pollock et al., Proc. Natl. Acad. Sci. USA 88:10480-10484, 1991; Furchgott, JAMA, 276:1186-1188 (1996)].

Concerning arterial blood pressure control, NO synthesized by the vascular endothelium has been shown to be very important for the regulation of the vascular tonus in mammals [Vallance et al., Cardiovasc. Res., 23:1053-1057 (1989)]. On the other hand, long-lasting vasodilation depends on the sustained increase of NO production by endothelial cells [(Huang et al., Nature, 377: 239-242 (1995); Shesely et al., Proc. Natl. Acad. Sci. USA, 93: 13176-13181 (1996)], which need the recycling of citrulline into argentine, the sole substrate for NO production in caveola, since these cells do not utilize arginine from other cellular compartments [Pendleton et al., J. Biol. Chem., 280: 24252-24260 (2005); Solomonson et al., J. Exp. Biol., 206:2083-2087 (2003)].

Accordingly, the pathway involving the enzymes AsS and NOS has a critical role in the regulation of endothelial, systemic, renal, and coronary hemodynamic functions, in platelet adhesion and aggregation, in cardiomyocyte hypertrophy, and in the proliferation of vascular smooth muscle cells, and in fibrosis [Govers and e-Rabelink, Am. J. Physiol. Renal Physiol., 280:F193-F206 (2001); Vallance and Chan, Heart, 85: 342-350 (2001); Huang et al., Nature, 377: 239-242 (1995)].

Among other mechanisms, maintenance of a higher level of NO should induce relaxation of blood vessels through the activation of Maxi K channels, causing a membrane hyperpolarization. This pharmaco-mechanical and electrophysiological mechanisms, which involve the ion channel Maxi K, are important factors in the vascular relaxation, induced by NO (Tanaka et al., J. Smooth Muscle Res., 40:125-153 (2004)].

Intracellular Ca²⁺ concentration [Ca²⁺]ᵢ is also related to the vascular relaxation through the Maxi K channels, since the increase of intracellular Ca²⁺ can lead to the activation of the Maxi K channels of the cell, if the channel belongs to the K⁺ channel family activated by Ca²⁺[(Tanaka et al., J. Smooth Muscle Res., 40:125-153 (2004)].

It is well known that NO influences the signaling of intracellular events, as the regulation of intracellular homeostasis of Ca²⁺, for instance. In fact, NO increases vascular [Ca²⁺]ᵢ by stimulating the inositol triphosphate mediated Ca²⁺ mobilization, by an increase in the accumulation of cytosolic Ca²⁺ through inhibition of the Ca²⁺ ATPase of the sarcoplasmic/endoplasmic reticulum, and by stimulating extracellular Ca²⁺ influx through the Ca²⁺ channels. In other words, the increase in NO generation increases Ca²⁺ signaling via [Ca²⁺]ᵢ, and regulates vascular contractility and tonus [Touyuz, Antioxid. Redox Signal., 7(9-10): 1302-1314 (2005)].

On the other hand, circulating effectors, like bradykinin, for instance, bind to endothelial cell receptors at the luminal surface, causing an increase in [Ca²⁺]ᵢ, which in turn activates the endothelial nitric oxide synthase (eNOS) through the calcium-calmoduline complex formation.

Therefore, through its direct or indirect effects via [Ca²⁺]ᵢ, and via the Maxi K channels, the endothelial NO is essential for the regulation of the cardiovascular homeostasis. Furthermore, together with prostacyclin, NO has a potent antiteratogenic property, and also an anti-thrombus resistance characteristic, by preventing platelet aggregation and cellular adhesion [Furchgott, JAMA, 276:1186-1188 (1996); Zhou e Frohlich, Am. J. Nephrology, 25:138-152 (2005)].

Several drugs and treatments, which increase the quantity of available NO, and/or increase the quantity of produced NO, have been developed in order to treat or prevent cardiovascular diseases, as well as other diseases related to NO deficiency. U.S. Pat. No. 6,447,768 describes the use of gene therapy which employs a vector containing the coding sequence of the NOS, to increase the availability of the enzyme, thus enhancing NO synthesis. Although promising, gene therapy is still not very well known, it is invasive, and inherent risks are high. On the other hand, described conventional therapies, usually based on combined therapies, employ several drugs simultaneously. U.S. Pat. No. 6,635,273 describes the combined therapy of cardiovascular diseases, using anti-oxidants, which indirectly stimulate NO synthesis through oxidation of L-arginine to L-citrulline, plus ACE inhibitors, and/or beta-blocking agents and/or antagonists of calcium channels.

Disadvantages of this therapy lie in the fact that anti-oxidants lead to slight alterations in the NO production, which, by themselves, would not be sufficient to treat hypertension efficiently, therefore needing a combined therapy with other anti-hypertensive agents that act on different targets.

Another indirect way to increase the production of endothelial NO in cardiovascular diseases would be by means of ACE inhibitors, since anti-hypertensive and cardioprotector effects of these inhibitors have been explained, at least partially, as consequence of the reduction of bradykinin degradation, which results in an increase of endothelial NO production [Zhang et al., X, J. Pharmacol. Exp. Ther., 288:742-751 (1999)], and enhanced endothelial function [Horning et al., Circulation, 95:1115-1118 (1997)]. This increase in NO production is, however, an indirect effect, and insufficient to treat and/or prevent adequately cardiovascular diseases. Furthermore, the inhibition of ACE can cause side effects because this enzyme is involved in other physiological processes [Zhang et al., X, J. Pharmacol. Exp. Ther., 288:742-751 (1999)].

More recently, Keefer and collaborators described (U.S. Pat. No. 4,954,526; U.S. Pat. No. 5,039,705; U.S. Pat. No. 5,155,137; U.S. Pat. No. 5,208,233; U.S. Pat. No. 5,405,919 e U.S. Pat. No. 6,949,530) the use of several compounds, which once hydrolyzed, or in the presence of acid, or once degraded release NO. These compounds release NO in an exogenous manner, and do not act on the synthesis of endogenous NO. Besides, these compounds present a strong disadvantage of representing a potential risk of undesired release of carcinogenic nitrosamines, after their decay and the release of NO.

The Brazilian patent application BR0400192 claims pharmaceutical compositions containing peptides extracted from the venom of the Bothrops jararaca snake, which act as agonists, partial agonists, antagonists or allosteric modulators of the acetyl choline receptor, which act on disorders caused by dysfunctions of cholinergic receptors. Yet another Brazilian patent application, BR0205449, claims the pharmaceutical compositions containing peptides extracted from the venom of the Bothrops jararaca snake, able to inhibit vasopeptidases, and named evasins, which were presented as alternatives to the pharmaceutical products class of angiotensin converting enzyme inhibitors, to be used as drugs to treat degenerative chronic diseases. As easily verifiable by a specialist in the field, none of the patent applications claim the participation of these naturally occurring peptides in the sustained production of NO, displaying the chemical and pharmaceutical properties of the present invention.

U.S. Pat. No. 3,819,831 relates to angiotensin converting enzyme inhibitors, which block the conversion of the decapeptide angiotensin I into the octapeptide angiotensin II, obtained from an extract fraction of the Bothrops jararaca venom. U.S. Pat. Nos. 4,105,776, 4,129,571, and 4,154,960 relate to synthetic derivatives of the amino acid proline, which include the medicament captopril (D-3-mercapto-2-methyl-1-oxopropyl-L-proline), a well-known active site directed ACE inhibitor, used to treat arterial hypertension, and its newer derivatives enalapril and lysinopril (U.S. Pat. No. 4,374,829), among others.

These ACE inhibitors are efficient in treating hypertension, in decreasing clinical events in high-risk patients with atherosclerosis, in enhancing left ventricular dysfunction, in diminishing clinical events after myocardial infarct, and in reducing morbidity and mortality in congestive cardiac insufficiency patients [Yusuf et al., N. Engl. J. Med., 342:145-153 (2000)].

However, in spite of the documented clinical efficacy of the ACE inhibitors, a substantial number of hypertensive patients are not adequately controlled with a monotherapy using ACE inhibitors, needing a combined therapy, which includes diuretic agents, beta-blocking agents, and/or calcium channel antagonists [Yusuf et al., N. Engl. J. Med. 342: 145-53 (2000)]. In spite of the facts just described, morbidity and mortality remain elevated in these patients [The Acute Infarction Ramipril Efficacy (AIRE) Study Investigators, Lancet, 342:821-828 (1993); Kober et al., N. Engl. J. Med., 333:1670-1676 (1995)].

Furthermore, in relation to the therapeutic efficacy of the ACE inhibitors, it is important to mention that the dose required is the one able to inhibit, in vivo, the conversion of angiotensin I in angiotensin II, and inactivate bradykinin. Thus, in order to obtain the anti-hypertensive effect, the doses are, for instance, 1000 fold higher than the anti-hypertensive doses used in the present invention. Knowing that the ACE has other physiological functions, not solely related to the kinin-angiotensin system, [Cotton et al., Selective inhibition of the C-domain of angiotensin I converting enzyme by bradykinin potentiating peptides Biochemistry 41: 6065-6071, 2002], it is understandable that these inhibitors cause so many side effects and adverse reactions, some of which can be severe, such as, angioneurotic edema, cutaneous eruption, dry cough, hypotension, blood cell dyscrasias, and erectile dysfunction.

Thus, the present therapeutic strategies are limited in their capacity of significantly modifying the course of several diseases, since they affect single independent targets and usually offer a temporary and incomplete benefit to the patients. A number of cardiovascular diseases, for instance, are multi-factorial illnesses that, ultimately, affect the regulation of the sustained NO biosynthesis; therefore, drugs acting on multiple targets that synergistically up-regulate NO biosynthesis, might be an efficient alternative to therapeutic strategies to come.

One of said deregulations is the one affecting the sustained production of NO, which cause arterial hypertension, for instance. Thus, the sustained production of NO is crucial for the treatment and the prevention of hypertension, as well as for the treatment and the prevention of other diseases involving NO deficiencies.

As can be observed in the state of the art, until the present, products developed to treat disorders related to NO deficiency present inconveniencies concerning their chemical nature, difficulties in obtaining them, efficacy and side effects.

The pharmaceutical compositions described in the present invention, containing an oligopeptide or a mixture of different proline rich oligopeptides herein described, are able to activate nitric oxide biosynthesis by multiple mechanisms, such as by activating the enzyme AsS, or by promoting an increase in the [Ca²⁺]_{I} in endothelial cells. Thus, diseases caused by NO deficiency that can be treated by the method herein described, include cardiovascular diseases, disorders of the nervous system, disorders of the gastrointestinal system, disorders of the immune system, disorders of the systemic, the renal and the coronary hemodynamics, neurodegenerative pathologies, pre-eclampsia, lymphocyte dysfunction during immune response and/or tumor growth, erectile dysfunction, and the control of germinative cell production. They can be used in humans as well as in veterinary clinics.

### OBJECTIVES OF THE INVENTION

In view of the exposed, the present invention has the objective of providing multiligand oligopeptides able to bind to diverse targets capable of increasing, synergistically, the production of NO in mammal cells by directly stimulating NO biosynthesis, be it by activating the argininosuccinate synthase (AsS) enzyme, and/or by increasing intracellular bivalent calcium ion (Ca²⁺) concentration.

Another objective of the present invention provides pharmaceutical compositions that employ the oligopeptides described in the present invention.

A third objective of the present invention is the use of the oligopeptides described in the present invention for manufacturing medicaments to be used in diseases involving NO deficiency.

An additional objective of the present invention provides methods of treatment and/or methods of prevention of pathologies involving NO deficiencies employing oligopeptides described in the present invention.

### BRIEF DESCRIPTION OF THE DRAWINGS

FIG. 1—SDS Page gel showing elution of one band of approximately 46 kDa which bound to the oligopeptide SEQ ID NO 7 after chromatography of mouse kidney extract though an affinity columns containing oligopeptide SEQ ID NO 7 as ligand.

FIG. 2—Western Blot of purified membrane and cytosolic proteins of mouse kidney. Proteins of two independent experiments were purified by affinity columns containing SEQ ID NO 7 peptide. Bands in lane 1 and 2 identify the AsS of membrane and cytosol, respectively, obtained in Experiment 1; bands in lanes 3 and 4 identify the AsS of membrane and cytosol, respectively, obtained in Experiment 2.

FIG. 3—Activation of the enzyme argininosuccinate synthase (AsS) by oligopeptide SEQ ID NO 8. Results are presented as absorbance (650 nm) as a function of the added quantity of SEQ ID NO 8 (in μM). Absorbance is related to the amount phosphate present in the reaction mixture, which in turn is related to the AsS activity. Consequently, the graph reflects the variation in enzymatic activity of the AsS as a function of the added oligopeptide amount.

FIG. 4—Activation of the enzyme argininosuccinate synthase (AsS) by peptide SEQ ID NO 8. Results are presented as absorbance (650 nm) as a function of the added amount of the enzyme AsS (in μg). Absorbance is related to the amount phosphate present in the reaction mixture, which in turn is related to the AsS activity. Consequently, the graph reflects the variation in enzymatic activity of the AsS as a function of the added enzyme amount. Rhombuses represent the absorbance in the presence of AsS. Triangles represent absorbance observed in the presence of AsS activated by the addition of peptide SEQ ID NO 8. Squares represent the absorbance observed in the presence of AsS and its specific inhibitor MDLA. Circles represent the absorbance in the presence of AsS activated by the addition of peptide SEQ ID NO 8, and in the presence of the specific inhibitor of AsS, MDLA.

FIG. 5—Increase in the intracellular concentration of free [Ca²⁺]ᵢ in SK-N-AS cells as a function of time, after addition of oligopeptides SEQ ID NO 1 and SEQ ID NO 8 peptide (lower panels). Upper panels show that there is no change in the intracellular concentration of calcium in non-stimulated cells and in cells treated with captopril.

FIG. 6—Nitrite generation as function of increasing concentrations of oligopeptide SEQ ID NO 8 (in μM): (A) in the intra- and extracellular milieu of endothelial cells; (B) in the intracellular milieu of neuroblastoma cells, presented as percentage increase in relation to the control.

FIG. 7—Determination of the levels of nitric oxide in cell cultures of C6 cells, incubated with diverse proline rich peptides. Identification of the peptides numbered in the graph: 1—basal level; 2—L-NAME (1 mM); 3—Nitroprussiate (1 mM); 4—SEQ ID NO 2; 5—SEQ ID NO 3; 6—SEQ ID NO 4; 7—SEQ ID NO 5; 8—SEQ ID NO 6; 9—SEQ ID NO 8; 10—SEQ ID NO 13; 11—SEQ ID NO 14.

FIG. 8—Bioavailability of the synthetic oligopeptide SEQ ID NO 8 labeled with ¹²⁵I in mouse tissues. Results are presented as the dose percentage of the labeled SEQ ID NO 8/¹²⁵I per mg of tissue, extracted 180 min after administration of the peptide.

FIG. 9—Total NOS activity (in pmol/mg×min) determined in the protein extract of Swiss mouse renal tissue treated with captopril, SEQ ID NO 8, L-NAME, HOE140, SEQ ID NO 8/L-NAME, and SEQ ID No 8/HOE140. All samples were compared to the control (non-treated animals). Comparison significances were: (♦) p<0.01, (#) p<0.05 in relation to the control, and (*) p<0.05 in relation to control/HOE140. Five mice were used in each assay (n=5). The graph follows the numbering: 1—Control; 2—Captopril; 3—SEQ NO ID 8; 4—Control/L-NAME; 5—SEQ NO ID 8/L-NAME; 6—Control/HOE140; 7—SEQ NO ID 8/HOE140.

FIG. 10—Evaluation of NO levels (μM) in total protein kidney extract incubated with peptide SEQ ID NO 8, in doses of nM, 1 μM and 10 μM. Controls are: basal level, nitroprussiate, NO donor as positive control, and L-NAME, a specific NOS inhibitor as negative control. The graph follows the numbering: 1—basal level; 2—Nitroprussiate (1 mM); 3—L-NAME (1 mM); 4—SEQ ID No 8 (10 nM); 5—SEQ ID No 8 (1 μM); 6—SEQ ID No 8 (10 μM).

FIG. 11—Effects of proline rich peptides and captopril on the mean arterial blood pressure (MAP) of normotensive and spontaneously hypertensive rats (SHR) as a function of time. In all panels the test for the conversion of angiotensin I into angiotensin II is shown, before and after administration of the anti-hypertensive compound, followed by determination of the mean arterial blood pressure during 360 minutes. Values represent the mean, and standard mean deviation of five animals for each dose are given (ΔMAP, mm Hg). In panel A 71 nmol/Kg of peptide SEQ ID NO 8 were administered to normotensive rats. In panel B 71 nmol/Kg peptide SEQ ID NO 8 were administered to SHR. In panel C 71 nmol/Kg of peptide SEQ ID NO 4 were administered to SHR. In panel D 71 nmol/Kg captopril were administered to SHR. In panel E 10 μmol/Kg captopril were administered to SHR.

FIG. 12—Dose-effect relation of peptide SEQ ID NO 8 on the mean arterial blood pressure on SHR. Doses of 3 nmol, 15 nmol, 71 nmol e 140 nmol/Kg body weight were administered to groups of 5 SHR. Values are presented as the mean (ΔMAP, mm Hg), and standard mean deviations of the anti-hypertensive activity are given for each dose.

FIG. 13—Anti-hypertensive effect of different proline rich peptides on the mean arterial blood pressure of SHR. The figure represents the mean values for ΔMAP (mm Hg), and the standard mean deviation of groups of five SHR are given, which received a dose of 71 nmol/Kg body weight of peptides SEQ ID NO 1, SEQ ID NO 4, SEQ ID NO 5, and SEQ ID NO 8. 5 SHR (controls) were injected saline solution.

FIG. 14—Effect of pentobarbitone on the anti-hypertensive activity of peptides SEQ ID NO 1, SEQ ID NO 4, SEQ ID NO 8 and captopril in SHR. Two groups of 25 SHR were tested, one being anesthetized with pentobarbitone, and the other conscious SHR, who received the same dose pentobarbitone 24 hours before. For each group (anesthetized and conscious) groups of 5 animals received doses of 71 nmol/Kg of peptides SEQ ID NO 1, SEQ ID NO 4, SEQ ID NO 8, and captopril (10 μmol/Kg). For both groups 5 SHR were injected with saline solution (controls). Values represent ΔMAP (mm Hg), and the standard deviation of the mean is given.

### DETAILED DESCRIPTION OF THE INVENTION

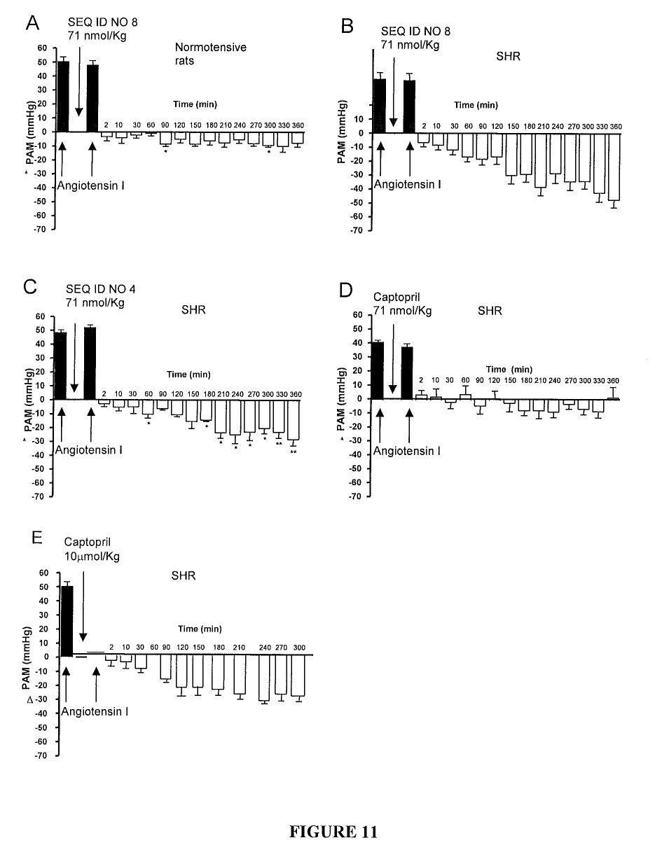

The present invention provides proline rich oligopeptides for the treatment of dysfunctions and conditions associated to nitric oxide (NO), such as hypertension, diabetes, thrombosis, angina, heart, and atherosclerosis. The inventors observed that several synthetic proline rich oligopeptides described in the present invention (Z-pros) reduce the mean arterial blood pressure of spontaneously hypertensive rats (SHR), but not of normotensive rats, when injected i.v. as a single dose of approximately 0.01 nmol/Kg to 900 nmol/Kg of body weight, preferentially between 0.1 nmol/Kg and 200 nmol/Kg body weight, more preferentially approximately 0.5 to 80 nmol/Kg of body weight. Assays performed in SHR showed that doses between 0.5 and 80 nmol/Kg of proline rich oligopeptides of the present invention were able to reduce the arterial blood pressure of these animals by 30 to 50 mm Hg, and that this effect lasted for over 6 hours. This type of mean arterial blood pressure reduction does not occur acutely, but sets in between approximately 60 and 120 minutes, preferentially 90 min after injection with the peptide of the present invention, as exemplified in FIG. 11 A-E.

In another embodiment of this invention, the lack of a hypotensor effect on normotensive rats is illustrated, but not restricted to the data present ed in Example 14. Accordingly, even receiving doses 1000 times higher than those that presented anti-hypertensive effects in SHR, normotensive rats do not suffer significant alterations in the mean arterial blood pressure.

The inventors also observed that the dose capable of producing the anti-hypertensive effect of the peptides of the present invention in SHR was at least three orders of magnitude lower than the peptide concentration required to inhibit the angiotensin converting enzyme (ACE) in vivo. In contrast to the peptides of the present invention, captopril, a site-directed inhibitor of the ACE, was only able to exert anti-hypertensive activity in a molar dose approximately 1000-fold higher, that is, at the dose capable of inhibiting the activity of the ACE in vivo (e.g. 10 μmoles/Kg, FIG. 11E). These observations, illustrated in Example 12, FIG. 11 A-E, refer to peptides SEQ ID NO 4 and SEQ ID NO 8, but by no means are restricted to the examples presented here. As can be widely found in the state of the art, the metabolic route employed by most medicines, for instance, to control arterial blood pressure is the direct inhibition of bradykinin degradation by ACE inhibitors. In fact, we show that the compounds described herein act through a completely different mechanism than that described for drugs that inhibit the ACE, and are presently being employed. Therefore, the present invention should waken pharmaceutical interest, since the ACE inhibitors present numerous side-effects, such as angioedema, renal dysfunction, cough, and hypotension, which are consequences of the ACE inhibition, and are widely described in the state of the art [Leeb-Lundberg et al., Pharmacological Rev., 57:27-77, (2005)].

The present invention also provides methods which allow associating the effects of the peptides herein described to the sustained production of nitric oxide (NO) by the kidneys. NO displays multiple biological effects, in particular those affecting the regulation of physiological functions, and pathological conditions. The inventors observed that peptides of the present invention selectively concentrate in the kidneys, when injected into mice. Example 8 illustrates, but is not restricted to, the bioavailability of peptide SEQ ID NO 8/¹²⁵I. When this peptide is injected intraperitoneally into mice, radioactivity rapidly concentrates in the kidneys, and the equivalent of approximately 15% of the concentration of the injected dose remains in this tissue for longer than 3 hours. Compared to other mice tissues, the kidneys display a concentration higher than 10 fold the concentration found in the other tissues per gram of tissue. This fact is surprising since peptides are usually very rapidly degraded by blood and tissue peptidases, mainly in renal tissue. It is known that, in vitro, proline rich peptides are resistant to proteolytic degradation. This resistance to degradation is reflected in the prevalence of integer peptides found in the urine of mice that were injected intraperitoneally with the peptides of this invention. As illustrated in Example 9, but not restricted to, it is evident that all peptides are excreted mostly in their integer form, with exception of peptide SEQ ID NO 1, which presents only one C-terminal proline (Example 9, Table 2).

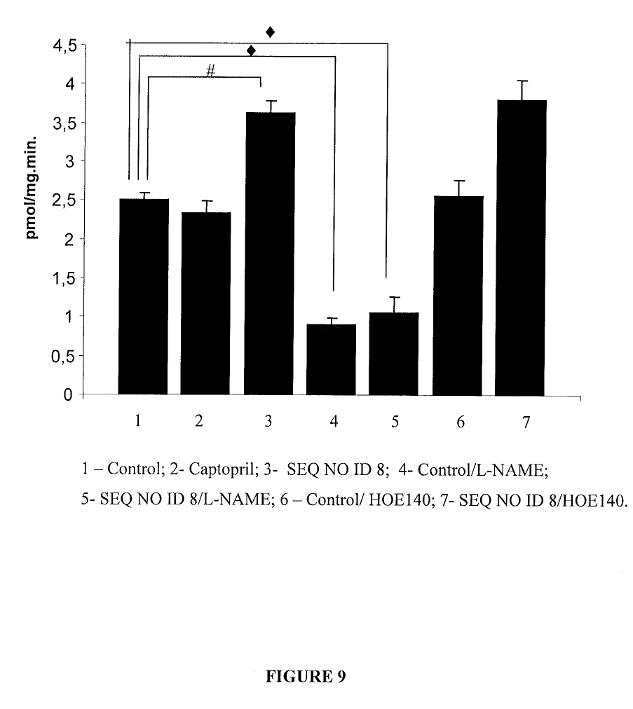

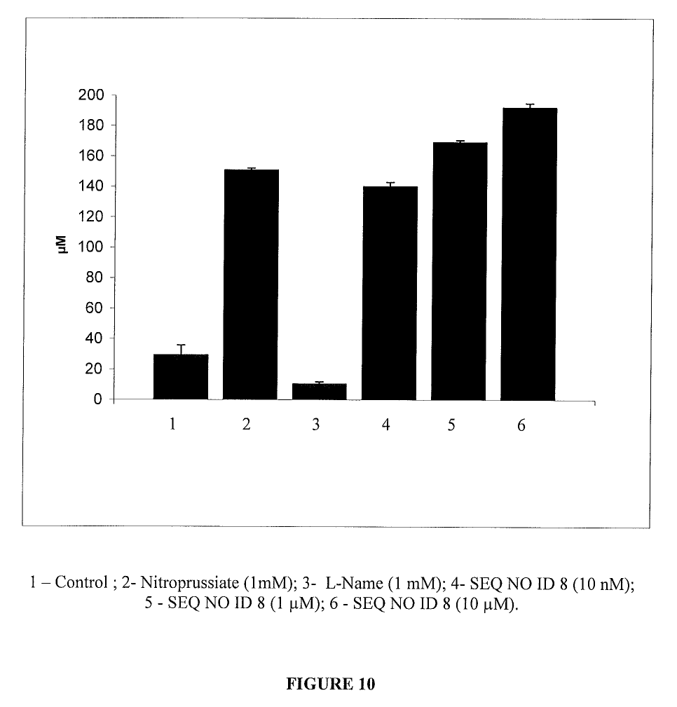

It is known that the kidneys play a central role in arterial blood pressure control, a fact that coincides with its high efficiency in NO production. Accordingly, in another aspect of the present invention, we show as an example, not restricting ourselves to this molecule, that approximately 0.1 to 5 mg/Kg body weight, preferentially 0.8 to 2.5 mg/Kg body weight, even more preferentially, approximately 1 mg/Kg body weight of peptide SEQ ID NO 8 was able to activate the mouse renal nitric oxide synthase (NOS) activity by approximately 60% (FIG. 9). No significant renal NOS activity was observed when animals were injected with captopril at a concentration of 0.2-3.5 mg/Kg of body weight. It became evident that bradykinin does not participate at this process, although it could intermediate the activation of NOS by the SEQ ID NO 8. Accordingly, the specific inhibitor of the B₂ receptor of bradykinin (HOE 140, 10 μg/Kg for 1 hour), for instance, was not able to prevent the increase caused in NOS activity by peptide SEQ ID NO 8. Corroborating with this result, we were able to show that 0.01 to 50 μmol, preferentially 0.5 to 2 μmol of peptide SEQ ID NO 8 increased nitrite production (generated from NO) by 5-fold in kidney homogenate of mice treated with peptide SEQ ID NO 8 (FIG. 10). These results are presented as examples, and for no means are restricted to the peptide that was used. Accordingly, this invention provides employing peptides of the present invention to increase renal production of NO, and activate NOS, which should significantly contribute to lower arterial blood pressure in hypertensive animals.

In another embodiment of this invention, peptide SEQ ID NO 8 was found capable of selectively binding to an enzyme present in the crude extract of mouse kidney, involved in the sustained production of NO. Using affinity chromatography with peptide SEQ ID NO 7 as ligand, Western blot and mass spectrometry allowed to identify the target protein of approximately 46 kDa corresponding to argininosuccinate syntase (AsS), an enzyme of the urea cycle, essential for the continuous delivery of NO to cells [Husson et al. Argininosuccinate synthetase from the urea cycle to the citrulline-NO cycle. Eur. J. Biochem. 270: 1887-1899 (2003)].

In another aspect of this invention, the AsS showed to be activated by peptide SEQ ID NO 8, maximum activation occurring at an approximate concentration of 0.05 to 100 μM, preferentially at a concentration of 1 to 5 μM of peptide SEQ ID NO 8. This activation which might reach 70-80%, is specific, since it is completely inhibited by the specific AsS inhibitor α-methyl-DL-aspartic acid (MDLA). Example 4 is presented as illustration, and does not restrict this characterization to oligopeptide SEQ ID NO 8.

Accordingly, the invention provides methods of utilizing the interaction of proline rich peptides, herein described, with the AsS, presenting themselves as alternative therapeutic agents, which use the activity of a target protein (AsS), not yet employed for therapeutic ends, as an endogenous pathway to increase sustained NO production. This aspect of the invention represents an unprecedented alternative for the treatment of dysfunctions caused by NO insufficiency.

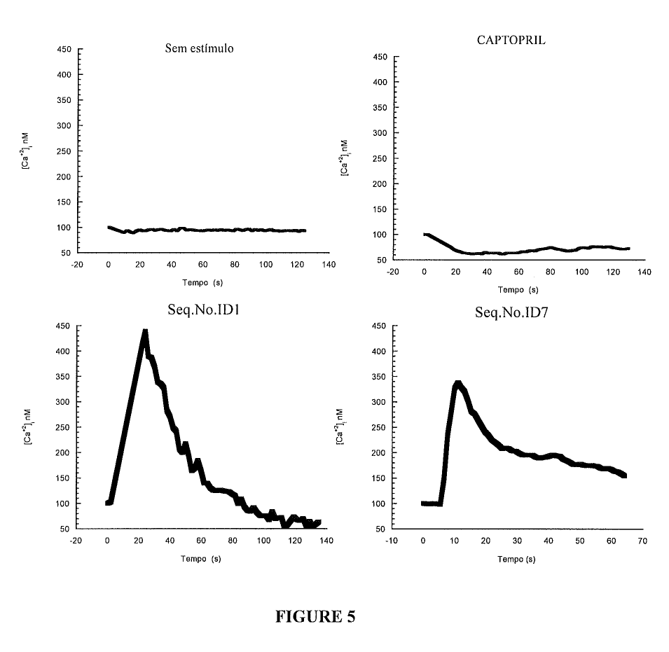

Proline rich oligopeptides may also be useful in the scope of the present invention to increase the intracellular concentration of Ca²⁺[Ca²⁺]ᵢ. Pathologies and dysfunctions associated to the insufficient production of NO might also result from disorders in processes that regulate the [Ca²⁺]_{I}, since this bivalent ion activates calmodulin, an “important regulator” of several cellular processes, which include activation of the endothelial (eNOS) and the neuronal NOS (nNOS). Deregulation of this process might aggravate cardiovascular, neuronal, endocrine and immune pathologies. Consequently, another embodiment of the present invention concerns activation of Ca²⁺ release in endothelial and nervous cells by the peptides of the present invention. Example 5, FIG. 5 illustrates how peptides SEQ ID NO 1 and SEQ ID NO 7 stimulate the [Ca²⁺]_{I} in nervous cells (SK-N-AS, human neuroblastoma), not restricting by any means the example to the peptides nor to the cells tested. Peptides SEQ ID NO 1 and SEQ ID NO 7, at concentrations of 0.1 to 100 μM, preferentially at concentrations 1 to 10 μM, were able to increase [Ca²⁺]ᵢ in SK-N-AS cells, while captopril (1 μM) did not cause any change in the [Ca²⁺]ᵢ. The increase of [Ca²⁺]ᵢ was instantaneous and transient, that is, immediately after addition of the peptides the response peak reached approximately 350 and 250 nM for the stimulation by peptides SEQ ID NO 1 and SEQ ID NO7, respectively, decreasing rapidly, and reaching a plateau 20 seconds after the peak.

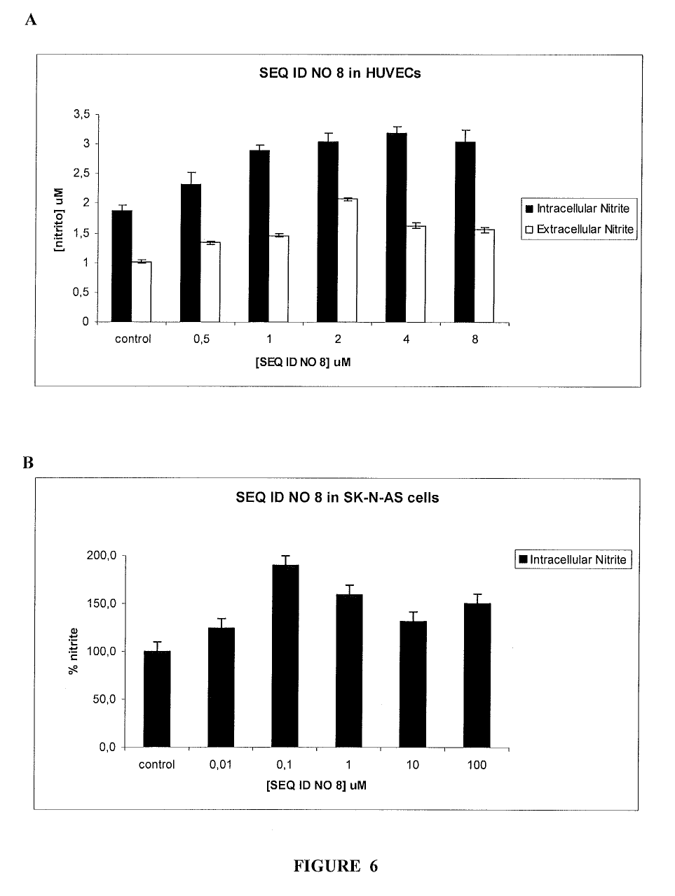

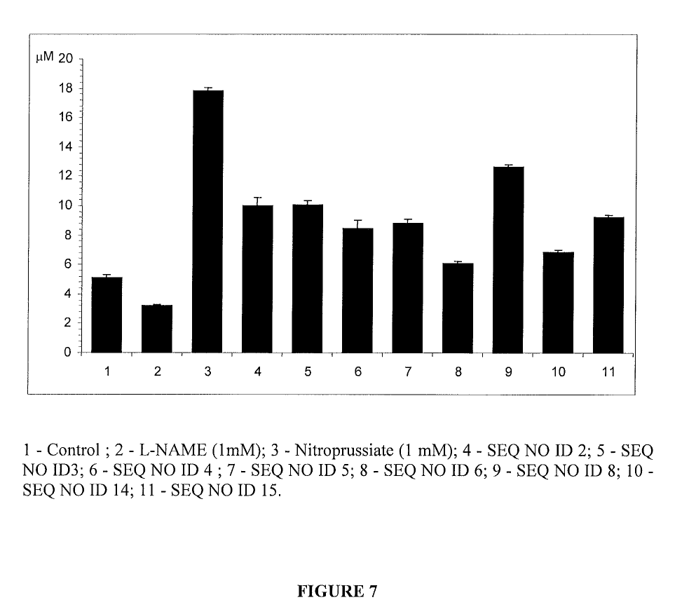

In another embodiment of the present invention, at concentrations of 0.01 to 500 μM, or preferentially between 0.1 and 100 μM, peptide SEQ ID NO 8 was able to increase NO generation in HUVEC and SK-N-AS cells by approximately 100% (Example 6, FIGS. 6A and 6B). Said activating effect on nitrite production can also be obtained in glia cells (glioma cells C6), using concentrations between 0.1 and 100 μM of proline rich peptides herein described, or preferentially in the concentrations between 1 and 10 μM. As examples, not restricting to peptides SEQ ID NO 2, SEQ ID NO 3, SEQ ID NO 4, SEQ ID NO 5, SEQ ID NO 6, SEQ ID NO 8, SEQ ID NO 14, and SEQ ID NO 15, activation varied approximately between 20 and 130%, peptide SEQ ID NO 8 being the most efficient (Example 8, FIG. 7).

Proline rich peptides described in the present invention are thus able of concentrating selectively in kidneys, of potentiating the activity of renal AsS, which increases the delivery of arginine for NO production, of activating renal NOS, and increase NO production in renal tissue, of increasing the [Ca²⁺]_{I} and NO production in nerve (SK-N-AS), endothelial (HUVEC), glial (glioma C6) cells. As such, peptides of the present invention converge their activities to act synergistically on the sustained production of NO. Said activities, which are carried out in concentrations up to 1000 fold lower than those of captopril, for example, should explain the long-lasting anti-hypertensive effect observed in hypertensive animals (SHR). Said peptides are therefore useful for the treatment and/or the prevention of pathologies involving NO deficiencies in mammals, comprising among others, cardiovascular diseases. Furthermore, the present invention demonstrates that said peptides do not act in normotensive animals, even if administered in high doses, conferring them safety and selectivity for pathologies, such as arterial hypertension.

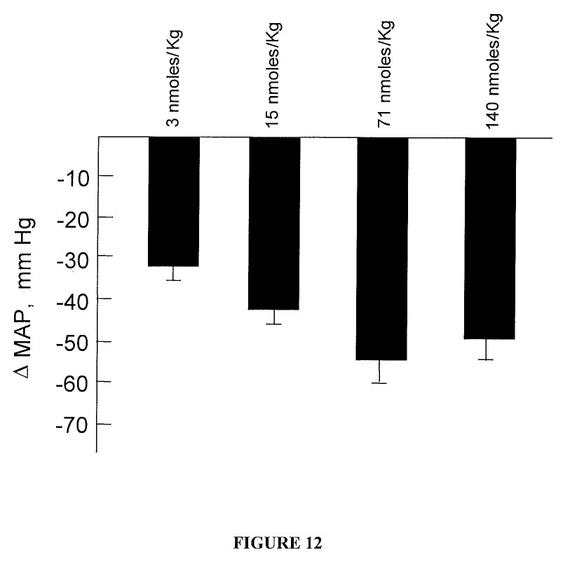

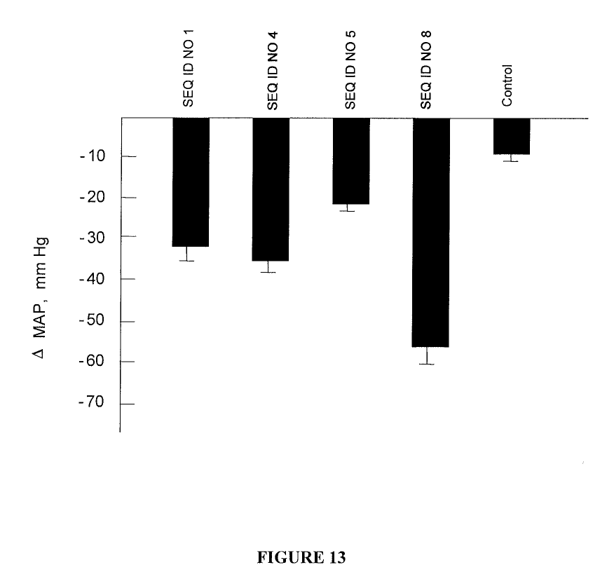

Another embodiment relates to the anti-hypertensive effect of the oligopeptides of the present invention in SHR, since quantitative and qualitative differences are manifested in said animals, when treated with different-sized oligopeptides, or with said oligopeptides displaying different amino acid sequences, such as those contained in the SEQ ID NO 1 to 18, displayed in the list presented in Table 1. The dose-effect relationship for one peptide, as for instance, for SEQ ID NO 8, is illustrated in Example 12, FIG. 12, but is not restricted to said peptide of the present invention. The optimum of the anti-hypertensive activity of said peptide occurs approximately at the dose of 71 nmoles/Kg of body weight, reducing this effect at higher, as well as at lower doses. Another aspect of the present invention is for the relationship of the amino acid sequences of the peptides of the present invention and the anti-hypertensive effect at the dose of 71 nmoles/Kg of body weight in SHR. Example 12, FIG. 13 does not restrict, merely illustrates, that the anti-hypertensive action of 4 peptides of the present invention on SHR is neither related to the peptide size, nor to the number of prolines/molecule.

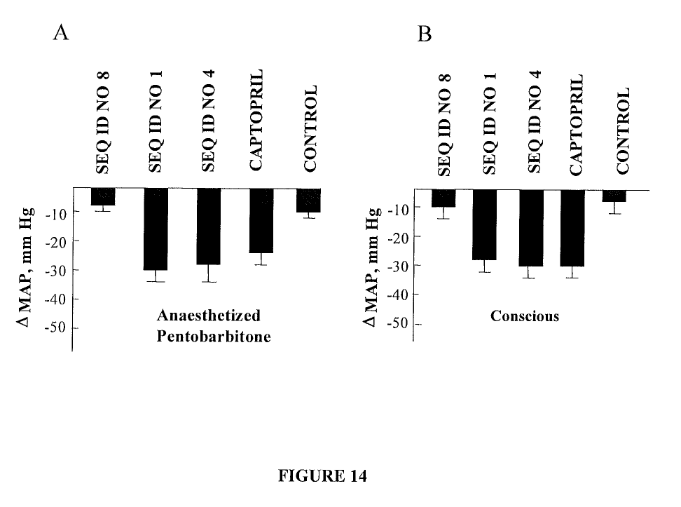

On the other hand, pentobarbitone is able to block the anti-hypertensive action of some of the peptides of the present invention on SHR, while the anti-hypertensive action of other peptides of the present invention, as well as of captopril, is not blocked by pentobarbitone. Said effect might last for at least 24 hours. FIGS. 14A and 14B of Example 13 are illustrations, but by no means restrict this effect to peptides depicted. This difference is important for the use in mammals of peptide SEQ ID NO 8, for example, since it restricts the use of said anti-hypertensive compound, which is susceptible to the action of pentobarbitone, by possibly not showing anti-hypertensive effect, when administered in association with (an)other medicament(s).

Taken together, the present invention provides evidences that demonstrate that proline rich peptides herein described act synergistically on regulatory mechanisms of the arterial blood pressure homeostasis, which have not yet been explored by any therapeutic class being employed in the treatment of this pathology.

Patients suffering from cardiovascular diseases have been taking advantage of drugs developed to act at a single molecular target. However, present pharmacological approaches are limited in their capacity of significantly modifying the course of the disease, offering a timely, incomplete benefit to patients. The innovation described in the present invention represents a new therapeutic strategy, and includes peptides acting on multiple cardiovascular and biochemical targets, causing improvement of the cardiovascular dysfunction, or of other pathologies, which depend on common transduction mechanisms, such as NO production. These multifunctional compounds can provide a higher efficacy, cause less side-effects and a better use of the drug in its function as a heart protector, consequently modifying the prognosis of the illness.

Furthermore, the synergistic mechanism displayed by the compounds of the present invention is advantageous in acting at much lower concentrations than anti-hypertensive compounds used today, of being resistant, as a whole, to degradation in vivo, and to maintain substantial concentrations of the active peptide in tissues for several hours, particularly in the kidneys. Specially, properties of the compounds of the present invention contribute to maintain for a long period of time, a reduction of the arterial blood pressure of hypertensive animals, a very important factor for the treatment of arterial hypertension.

Consequently, peptides of the present invention can be used to treat cardiovascular pathologies in humans, particularly arterial hypertension. Since the anti-hypertensive effect of the Z-pros are obtained at doses much lower than those needed for effectiveness of the ACE inhibitors (Example 11), it is expected that peptides described in the present invention do not produce side-effects caused by the increase in bradykinin concentration, such as cough, angioedema, disorder in blood cell production, accidents with patients submitted to extracorporial circulation, pro-angiogenic effects, etc, which are risk factors for the patient's health and life. These and other effects of blocking the ACE have been described of patients taking captopril, and its derivatives like enalapril and lysinopril. [Leeb-Lundberg et al., Pharmacological Rev., 57:27-77, (2005)].

According to the present invention, oligopeptides herein described present between 5 and 13 amino acid residues, displaying between approximately 600 and 1.500 Da molecular mass, and are described by identifying sequences 1 to 18 in Table 1.

Oligopeptides of the present invention might present chemical modifications at their amino-terminal portion, or at their carboxy-terminal portion, or at both ends of the oligopeptide, said modifications being introduced during the synthesis process of said oligopeptide. Said modification is performed either to endow higher stability to the oligopeptide, or to convert said oligopeptide in a biomarker. Said modification might be of any kind, as well as any labeling group, known to the state of art, might be employed, such as acetylation, biotinylation, synthetic amino acids such as hydroxyproline, and labeling with fluorophor groups.

Proline rich peptides of the present invention are synthetic and may be prepared by a variety of methods known to the state of the art. In the case of the present invention, all peptides were obtained by solid-phase synthesis. Said techniques have been described by Stewart and Young [in Solid-phase peptide synthesis, Freeman & Co., Ca, USA (1969)], and are exemplified in U.S. Pat. No. 4,105,603. The fragment condensation method for peptide synthesis is exemplified in U.S. Pat. No. 3,972,859. The technique utilized here was described to employ fluorenylmethoxycarbonyl (Fmoc) as protecting agent. Other available synthesis procedures are exemplified in U.S. Pat. No. 3,842,067 and U.S. Pat. No. 3,862,925.

A chemical group selected to be bound to the amino-terminal group of the peptide (group Z) was introduced using activating reagents. Satisfactory examples of activating reagents are carbodiimdes, such as N,N′-diisopropylcarbodiimide and N-ethyl-N′-(3-dimethylaminopropyl)carbodiimide. Other activating reagents and their use in peptide coupling have been described by Schoder and Lubke [in The Peptides, 1:72-75, Academic Press, NY, USA (1965)] and Kapoor [J. Pharm. Sci., 59:1-27 (1970)].

Finally, peptides bound or not to group Z, are purified by conventional methods, which include, among others, high performance liquid chromatography (HPLC). Purity and identity of said peptides are confirmed by amino acid composition analysis, mass spectrometry, analytic HPLC, among others.

Synthetic peptides may be purified by several methods described in the state of the art. Preferentially, the final deprotected peptide may be purified by HPLC in a reverse phase column, C-18, for instance, using a two solvent system: (A) trifluoroacetic acid (TFA)/H2O and (B) TFA/acetonitrile (ACN)/H2O in different proportions. An analytic HPLC system in reverse phase column moved by solvent system with a binary gradient coupled to a UV-VIS detector or a fluorescence detector verifies the purity of the peptide. The masses of purified peptides can be determined by mass spectrometry and/or by Edman degradation sequencing of the peptide. Its concentration in solution may be determined by amino acid analysis after acid hydrolysis, followed by derivatization with a fluorescent marker. Analysis of derivatized amino acids may be monitored, for instance, by fluorescence measurements. Chemical characteristics of purified synthetic products, presented in Table 1, confirm homogeneity of the peptides used in the present invention.

Another embodiment of the present invention relates to a pharmaceutical composition containing a proline rich oligopeptide, or a mixture of proline rich oligopeptides, capable of activating NO synthesis in cells of mammals, as well as, able to promote an increase in bivalent calcium ions (Ca²⁺) in the intracellular milieu, and its pharmacologic acceptable salts, besides adjuvant agents, such as excipients, diluents, or solvents. Said pharmaceutical composition of the present invention should be used in manufacturing medicaments for the treatment or the prevention of diseases caused by deficiency in the NO production, such as cardiovascular diseases, disorders of the nervous system, disorders of the gastrointestinal system, disorders of the immune system, control of germinative cell production, disorders of the systemic, renal and coronary hemodynamics, neurodegenerative pathologies, pre-eclampsia, lymphocyte dysfunction during immune response and/or tumor growth, and erectile dysfunction in mammals.

Peptides herein described can also be associated to other medicaments, in order to improve or complement the desired therapeutic effects.

Said pharmaceutical composition should contain between 0.05 μg and 10 mg of an oligopeptide, or of a mixture of different proline rich oligopeptides herein described, preferentially, said pharmaceutical composition should contain 0.5 μg to 0.005 mg of an oligopeptide, or a mixture of proline rich oligopeptides, and even more preferentially said pharmaceutical composition should contain 0.1 μg to 0.01 mg of an oligopeptide, or of a mixture of proline rich oligopeptides.

Another embodiment of the present invention relates to the use of proline rich peptides able to activate the biosynthesis of nitric oxide (NO) in the treatment of disorders caused by NO deficiencies in mammals. Since proline rich oligopeptides of the present invention are able to promote the biosynthesis of NO by means of multiple mechanisms, such as by activating the enzyme argininosuccinate synthase (AsS), or by increasing the intracellular concentration of the bivalent ion of calcium (Ca²⁺), said peptides are useful for the treatment of disorders caused by NO deficiency in the organism, as for instance, erectile dysfunction, cardiovascular diseases, disorders of the nervous system, disorders of the gastrointestinal system, disorders of the immune system, control of germinative cell production, disorders of the systemic, renal and coronary hemodynamics, neurodegenerative pathologies, pre-eclampsia, lymphocyte dysfunction during immune response and/or tumor growth in mammals.

The last embodiment of the present invention relates to the method of treatment of diseases caused by NO deficiency in animals, based on the administration to said animal of a pharmaceutical composition containing an oligopeptide, or a mixture of different proline rich oligopeptides, capable of activating the biosynthesis of nitric oxide.

Diseases caused by NO deficiency in animals which can be treated by the method herein described include cardiovascular diseases, disorders of the nervous system, disorders of the gastrointestinal system, disorders of the immune system, control of germinative cell production, disorders of the systemic, renal and coronary hemodynamics, neurodegenerative pathologies, pre-eclampsia, lymphocyte dysfunction during immune response and/or tumor growth, and erectile dysfunction, being able to be used by humans, as well as in the veterinary clinic.

The following examples, describing results obtained in assays performed with the Z-pro peptides of this invention, are offered for illustrative purpose only, and are not intended to limit the scope of the present invention in any way.

All patent and literature references cited in the present specification are hereby incorporated by reference in their entirety.

### EXAMPLES

### Example 1

### Preparation of the Synthetic Z-Pro Peptides

Briefly, peptides were synthesized in an automatic synthesizer PSSM8 (Shimadzu Co., Japan), in which sequential additions of amino acid residues, protected at the N-alpha with the base-labile group fluorenylmethoxycarbonyl (Fmoc), are made to an insoluble polymer support (Proline-2-Chlorotrityl resin). After removal of the Fmoc protecting group, the next protected amino acid is added using either a coupling reagent or a pre-activated amino acid derivative. The resulting peptide is attached to the resin, by means of a linker, through its C-terminal amino acid, and subsequently cleaved to yield an acidic peptide. Cleavage of the protecting N-alpha Fmoc group was performed with piperidine. Afterwards trifluoroacetic acid (TFA) is used for the final cleavage of the peptidyl resin, and deprotection of the side-chain, protected by the Fmoc-group. The peptide was purified by reverse phase liquid chromatography, and the identity and the purity of the peptide were analyzed by high pressure liquid chromatography (HPLC), and confirmed by mass spectrometry.

### Example 2

### Purification and Characterization of the Peptides

The synthetic peptides of the present invention were purified by high pressure liquid chromatography (HPLC). The final deprotected peptide was purified in an Econosil C-18 column (10μ, 22.5×250 mm) and a two-solvent system: (A) trifluoroacetic acid (TFA)/H₂O (1:1000), and (B) TFA/acetonitrile (ACN)/H₂O (1:900:100). Columns were eluted at a flow rate of 5 ml/min with a 10 (or 30)-50 (or 60) % gradient of solvent B over 30 or 45 min. The analytical HPLC system used for purity verification was a binary system from Shimadzu with an Ultrasphere C-18 column (5μ, 4.6×150 mm), coupled to a SPD-10AV Shimadzu UV-VIS detector, or to a Shimadzu RF-535 fluorescence detector. Elution was performed with solvents A and B described above, at a flow rate of 1 ml/min and a 10-80% gradient over 20 min. The eluates were monitored by absorbance at 220 nm, and/or by their fluorescence at λₑₘ 420 nm extinction, after excitation at λₑₓ 320 nm. The masses of purified peptides were determined by MALDI-TOF spectrometry (Ettan MALDI-TOF/Pro, Amersham Biosciences, Sweeden), and/or peptide sequencing (PPSQ-23, Shimadzu, Tokyo, Japan). After acid hydrolysis, concentration of the peptides was determined by amino acid analysis, performed in a HPLC system by Shimadzu, following OPA-derivatization monitored by fluorescence at 450 nm emission, and 350 nm excitation.

Characterization data of the synthetic peptides of the present invention are given in Table 1, below.

TABLE 1

| | Amino acid | Mass | Retention |
|---|---|---|---|
| Z-pro SEQ ID | Sequence (1 | sepectrometry | time |
| NO | letter code) | [M + H]⁺ | (min) |
| SEQ ID NO 1 | <EKWAP | 612.1 | 12.6 |
| SEQ ID NO 2 | <EKWAHyp | 628.2 | 12.2 |
| SEQ ID NO 3 | <ESWPGP | 654.2 | 12.9 |
| SEQ ID NO 4 | <EDGPIPP | 706.7 | 13.0 |
| SEQ ID NO 5 | <EWPRPQIPP | 1101.5 | 13.2 |
| SEQ ID NO 6 | <EYWPGPNIPP | 1151.5 | 13.3 |
| SEQ ID NO 7 | ENWPHPQIPP | 1214.6 | 13.4 |
| SEQ ID NO 8 | <ENWPHPQIPP | 1196.5 | 15.0 |
| SEQ ID NO 9 | Cy3-ENWPHPQIPP | 1680.6 | 15.2 |
| SEQ ID NO 10 | Bio-NPWHPQIPP | 1339.2 | 15.2 |
| SEQ ID NO 11 | Abz-NWPHPQIPP | 1204.5 | 14.5 |
| SEQ ID NO 12 | NWPHPQIPP | 1085.3 | 12.0 |
| SEQ ID NO 13 | <EARPPHPPIPP | 1189.2 | 14.7 |
| SEQ ID NO 14 | <EWGRPPGPPIPP | 1281.6 | 15.3 |
| SEQ ID NO 15 | <EGGWPRPGPEIPP | 1370.6 | 15.6 |
| SEQ ID NO 16 | PHPQIPP | 785.4 | 11.3 |
| SEQ ID NO 17 | ARPPHPPIPP | 1078.7 | 10.8 |
| SEQ ID NO 18 | WGRPPGPPIPP | 1171.1 | 13.6 |

P.S.: Z-pro, Proline Rich Oligopeptide, wherein Z is absent, or pyroglutamic acid (<E), Cy3, 2-amino benzoic acid (Abz), biotine (Bio)

### Example 3

### Identification of the Molecular Target Argininosuccinate Synthase (AsS)

Starting material were the cytosolic and plasma membrane crude extracts of mouse kidney cells homogenized in the buffer 10 mM Tris-HCl pH 7.5, 25 mM saccharose, 1 mM EDTA, and 1 mM PMSF (cytosolic fraction), or 1% Triton X-100 (plasma membrane fraction). Supernatants were dialyzed in 0.2 M NaHCO₃, 0.5 M NaCl, pH 8.3, and separately applied to the affinity chromatography column Hitrap NHS-activated (GE Healthcare) coupled to 5 mg of peptide SEQ ID NO 7.

Preparing affinity columns. Columns were washed three times with 2 ml 1 mM HCl before homogeneous preparations of SEQ ID NO 7 (5 mg in 1 ml 0.2 M NaHCO₃, 0.5 M NaCl, pH 8.3) were applied. After 30 min incubation at room temperature, peptides had been coupled, and the column was inactivated with buffer A (0.5 M ethanolamine, 0.5 M NaCl, pH 8.3), and washed with 0.2 M NaHCO₃, 0.5 M NaCl, pH 8.3, and stored at 4° C. in 50 mM Na₂HPO₄, 0.1%, NaN₃, pH 7.0. The columns were called HiTrap-SEQ. ID NO 7. A control column (without the peptide) was prepared by incubating the HiTrap NHS-activated HP with buffer A (Hi Trap-control).

Preparing renal extracts. Balb-c mice, weighing approximately 30 g, were anaesthetized with 50 μl 10% ketamine and 2% xylazine (1:1), and were submitted to intracardiac perfusion (infusion through the left ventricle and out-flow through the right atrium) with 20 ml saline (0.9% NaCl) and 0.01% sodium heparin with a flux of 4 ml/min. The kidneys were immediately removed and weighed, and for each 1 g of tissue, 1 ml buffer was added (10 mM Tris-HCl, 25 mM saccharose, 1 mM EDTA, and 1 mM PMSF, pH 7.5). Kidneys were minced in a tissue homogenizer (Polytron PT MR 3000, Kinematic AG, Littau), and centrifuged at 29.000 rpm for 35 minutes at 4° C. The supernatant containing the cytosolic proteins was stored, while the pellet was resuspended in the same buffer described above, containing 0.1% Triton X-100. The homogenate was centrifuged at the same conditions as above. The supernatant contained the membrane proteins.

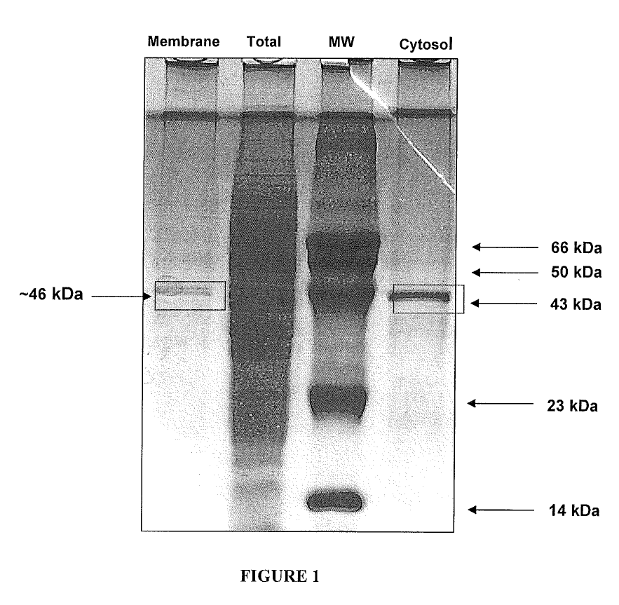

Affinity chromatography and analysis of the retained proteins by SDS-PAGE. HiTrap-SEQ ID NO 7 columns were equilibrated with two volumes of 20 mM Tris-HCl buffer, pH 8.0. Cytosolic and membrane fractions of the renal extract (100 mg/ml total protein) were applied to separate columns (1 ml/min flow rate), and washed with ten volumes of 20 mM Tris-HCl, pH 8.0. Proteins having affinity to SEQ ID NO 7 peptides were eluted with 100 mM glycine, 0.5 M NaCl buffer, pH 3.0, or, alternatively, by competition, with 5 mg/ml peptide SEQ ID NO 8 in 10 mM Tris-HCl, 25 mM saccharose, 1 mM EDTA, 1 mM PMSF, pH 7.5. Eluates were dialyzed in 10 mM NH₄HCO₃, pH 8.0 for 12 hours at 4° C. Protein concentration (mg/ml) was determined by the method of Bradford (Bradford, M. M. A rapid and sensitive method for the quantitation of microgram quantities of protein utilizing the principle of protein-dye binding. Anal. Biochem. 72: 248-254, 1976) with the Protein Assay reagent from BioRad. Bovine albumin was used as a standard, and measurements were performed at 595 nm in a spectrophotometer. Eluates were concentrated to 100 μl volume by centrifugation in vacuum, and a 5 μl aliquot was subjected to electrophoresis in a SDS-polyacrylamid gel, as described by Laemmli (Laemmli U. K. Cleavage of structural proteins during the assembly of the head of bacteriophage T4. Nature 227: 680-685, 1970). Results are presented in FIG. 1.

Characterization of the Retained Proteins

Mass spectrometry. The main band was cut out of the gel, fragmented and transferred to a 40% ethanol solution of 75 mM NH₄HCO₃. Several one hour incubations at room temperature were needed to destain the gel. Proteins contained in the gel were reduced with 5 mM DTT (dithiothreitol) in 25 mM NH₄HCO₃ for 30 min at 60° C., followed by alkylation with 55 mM iodine acetamide in 25 mM NH₄HCO₃ for 30 minutes at room temperature in the dark. One wash with 25 mM NH₄HCO₃ and another with acetonitrile were performed. Dehydration of the gel fragments was achieved by three 10 minute washes with acetonitrile, followed by drying in vacuum for 10 minutes. The gel was rehydrated in a solution containing 40 μg/ml trypsin (Trypsin Sequence Grade, Sigma) in 50 mM NH₄HCO₃ for 45 min on ice, and an incubation of 16 hours at 30° C. Protein fragments were extracted from the gel by the addition of 50 μl of 50 mM NH₄HCO₃ and a 10 min ultra sound bath, followed by the addition of 50 μl acetonitrile. The procedure was repeated three times. Eluates (extracted peptides) were dried in vacuum and resuspended in acetonitrile for mass spectrometry analysis by MALDI-TOF (Ettan MALDI-TOF, GE Healthcare). The protein band was identified as being the enzyme argininosuccinate synthase (AsS).

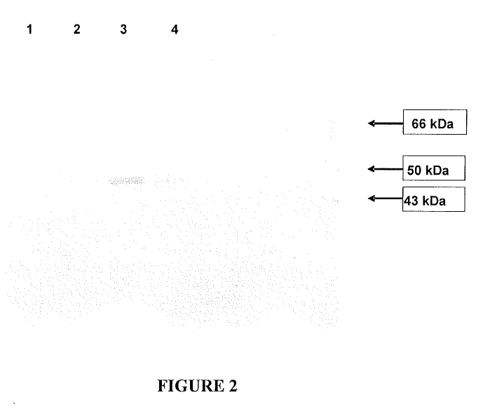

Western Blot. Following electrophoresis, proteins were electrotranferred to a nitrocellulose membrane using transfer buffer (380 mM Tris-HCl, 180 mM glycine, 20% methanol), under constant voltage of approximately 30V for 12 hours. After the transfer procedure, the membrane was stained with the Ponceau solution for 5 minutes to allow visualizing the bands, and determining their approximate molecular masses. Excess Ponceau solution was removed by washes with distilled water, and the membrane was incubated in a solution of 5% BSA (bovine serum albumine) in 0.05% TBS-Tween (TBS-T) and 0.02% azide for 1 hour at room temperature. The BSA solution was discarded, and the primary monoclonal anti-AsS antibody (BD Transduction Laboratories) diluted in TBS-T buffer (1:500, as recommended by the supplier) was added, and incubation followed for 1 hour at room temperature. The antibody solution was removed, and the membrane was washed three times with TBS-T for 10 minutes at room temperature. The secondary antibody, anti-mice IgG conjugated to alkaline phosphatase (Promega), diluted 1:7500 in TBS-T buffer was added. After a 1 hour incubation at room temperature, the membrane was washed three times with TBS-T buffer for 10 minutes, and the developer solution was added (solution AP [5 M NaCl, 1 M Tris-HCl, pH 9.5, 1 M MgCl₂] with BCIP (5-bromo-4-chloro-3-indolyl phosphate) and NBT (nitro blue tetrazolium), which allowed visualizing the band of interest. Results are presented in FIG. 2.

### Example 4

### Potentiation of the Argininosuccinate Synthase Activity (AsS)

Activation of the AsS enzyme of Example 3 by peptide SEQ ID NO 8 was quantified in vitro according to Hao et al. [Hao G., Xie L., Gross S. S. Argininosuccinate synthetase is reversibly inactivated by S-nitrosylation in vitro and in vivo. J. Biol. Chem., 279:36192-36200 (2004)]. This method is based on measuring the quantity of phosphate generated from ATP, when pyrophosphatase is added to the reaction medium. Since the activity of the pyrophosphatase relates directly to the enzymatic activity of the AsS, the quantity of generated pyrophosphate is a direct measurement of the AsS activity.

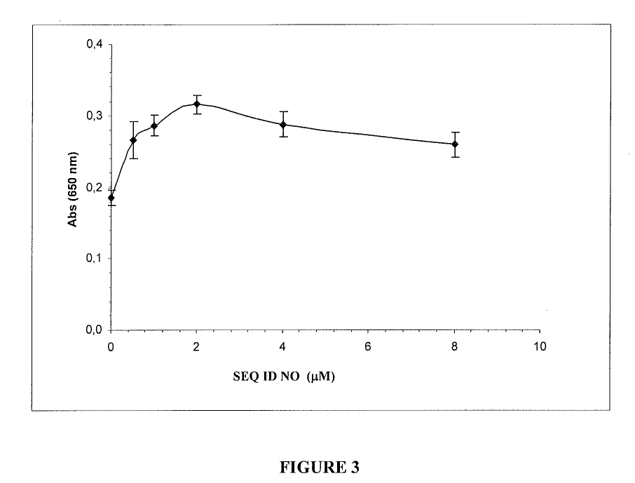

The AsS enzyme (1 μg) was added to the reaction mixture containing 20 mM Tris-HCl, pH 7.8, 2 mM ATP, 2 mM citrulline, 2 mM aspartate, 6 mM MgCl₂, 20 mM KCl, and 0.2 units of pyrophosphatase in a final volume of 200 μL in 96 well microplates. Increasing quantities of the SEQ ID NO 8 peptide (0.5, 1 to 8 μM) were added, and samples were incubated at 37° C. for 60 minutes. The reaction was interrupted by the addition of 200 μL of ammonium molybdate buffer (10 mM ascorbic acid, 2.5 mM ammonium molybdate, 2% sulphuric acid). Resulting phosphate was determined in the samples by spectrophotometry (absorbance at 650 nm), using a standard curve obtained with inorganic phosphate. Results are presented in FIG. 3.

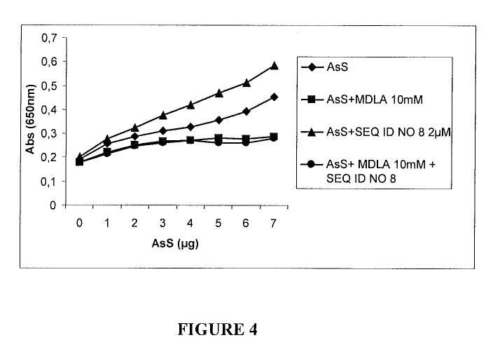

Specificity of the enzymatic activity was demonstrated in an assay, in which the AsS enzyme was added in increasing concentrations (1-8 μg) to the reaction medium described above, containing 2 μM of peptide SEQ ID NO 8. The production rate of phosphate (enzymatic activity of the AsS) was activated up to approximately 2.5 fold upon addition of 2 μM peptide SEQ ID NO 8. This activity was completely blocked by the specific AsS inhibitor MDLA (alpha-methyl-DL-aspartic acid) [Shen L. J., Beloussow K., and Shen W. C. Accessibility of endothelial and inducible nitric oxide synthase to the intracellular citrulline-arginine regeneration pathway. Biochem. Pharmacol. 69: 97-104, 2005]. These results are presented in FIG. 4.

### Example 5

### Increase in Free Intracellular Ca²⁺ Concentration

In these experiments the variation in free intracellular calcium [Ca²⁺]ᵢ concentration was measured in SK-N-AS cells (human neuroblastoma, ATCC No. CRL-2137) after addition of 1 μmolar of peptides SEQ ID NO 1 or SEQ ID NO 8, or of 1 μmolar captopril. Cells were kept in DMEM (Dulbecco Modified Medium), supplemented with 10% fetal bovine serum (FBS) and 1% non-essential amino acids, at 5% CO₂ atmosphere, at 37° C., and 95% relative humidity. Cells were treated with trypsin, and replicated at adequate cell concentrations.

Measurements of variation in the concentration of free [Ca²⁺]ᵢ were performed with a confocal microscope LSM 510 (Zeiss, Jena, Germany), as described by Martins et al., (Martins et al., Neuronal differentiation of P19 embryonal carcinoma cells modulates kinin B2 receptor gene expression and function. J. Biol. Chem. 280: 19576-19586, 2005). Approximately 5×10⁴ SK-N-AS cells were plated on 60 mm plates 24 hours prior to measurement, and then treated with 4 μM fluo-3 AM (Invitrogen Corporation) in 0.5% DMSO and 0.1% non-ionic surfactant pluronic acid F-127 for 30 minutes at 37° C. Cells were washed with DMEM containing 10% FBS, and transferred to defined medium (5 μg/ml insulin, 30 μg/ml transferrin, 20 μM ethanolamine, 30 nM sodium selenite, 1 μM sodium pyruvate, 1% non-essential amino acids, 1 mM glutamine, 100 μg/ml penicillin, 100 μg/ml streptomycin, 10 mM HEPES pH 7.4 in DMEM). The fluorescent fluo-3 AM was excited at 488 nm, and the fluorescence measured at 526 nm.

Variations in the [Ca²⁺]ᵢ were measured in non-stimulated cells, and in cells stimulated with peptides SEQ ID NO 1 or SEQ ID NO 8, or with captopril (Sigma Aldrich). Increase of fluorescence was monitored for 2 minutes, and images were acquired in 256×256 pixels every second. At the end of each experiment, 5 μM ionophore (4-Br-A23187) were added, followed by 10 mM EGTA, or 20 μM digitonin to determine maximum and minimum fluorescence, Fₘₐₓ and Fₘᵢₙ respectively, as described by Grynkiewicz et al., (1985).

Intracellular calcium concentrations [Ca²⁺]ᵢ, related to fluorescence (F) values obtained after addition of peptides SEQ ID NO 1 or SEQ ID NO 8, were calculated with the equation: [Ca²⁺]=K_{d}(F−Fₘᵢₙ)/(Fₘₐₓ−F), assuming K_{d}=450 nM for fluo-3 AM (Hallett et al., 1990). Results are presented in FIG. 5.

### Example 6

### Activation of NO Production in Human Endothelial and Neuroblastoma Cells

HUVEC cells (human umbilical vessel endothelial cells) were obtained from umbilical vessels, a gift from the University Hospital of the University of São Paulo. Vessels were washed externally with ethanol 96%, and after a diagonal cut at the end of the vessel, a catheter was introduced into the vessel vein, and attached to a three way valve. The vein was washed with 20 ml sterile saline, and the other end of the vessel was tied. One milliliter of a collagenase type IV solution was introduced (0.2 mg/ml/cm of umbilical vessel). The vessel was transferred to a Petri dish (10 cm²), and incubated for 15 minutes at 37° C. Cells contained inside the cord were removed by massage, and transferred to a sterile tube. Bovine fetal serum was added to a final concentration of 10%, and the suspension was centrifuged for 10 minutes at 3.000 g at 4° C., and the cell pellet was resuspended in 2 ml complete medium (40% Medium 199, 40% DMEM, 18% bovine fetal serum, 1% L-glutamine, 1% penicillin/streptomycin). The cell suspension was transferred to a bottle, previously treated with a 1% gelatin solution (30 min at 4° C.), and 5 mL complete medium was added. The bottle was incubated at 37° C. at 5% CO₂ atmosphere. The medium was exchanged after 12 hours, and later, every third day.

Human neurobalsotma cells (SK-N-AS, ATCC no. CRL-2137) were kept in DMEM, supplemented with 10% FBS (fetal bovine serum) and 1% non-essential amino acids, at 5% CO₂ atmosphere, at 37° C., and 95% relative humidity. Cells were treated with trypsin, and replicated when an adequate cell concentration had been reached. The medium was exchanged after 12 hours, and later, every third day.

The method of Feelisch et al. (Feelisch et al., Concomitant S-, N-, and heme-nitros(yl)ation in biological tissues and fluids: implications for the fate of NO in vivo. FASEB J 16: 1775-1785, 2000) was employed to analyze NO production in the endothelial and neuronal cells described above.

Cells were grown in Petri dishes to 80% confluence. Peptide SEQ ID NO 8 was added in the concentration range of 0-8 μM to HUVEC cells, and 0-100 μM to SK-N-AS cells, and incubation continued for 24 hours. Subsequently, 1 ml of medium was retrieved (extracellular medium), and replaced by 1 ml 10 mM N-ethylmaleimide. Cells were washed in PBS, and lyzed in RIPA buffer (50 mM Tris pH 7.5, 150 mM NaCl, 1% Nonidet p-40 (NP-40) and 0.5% Na-desoxycholate, 0.1% SDS, 1 mM DTPA, and 10 mM N-ethylmaleimide). Lysats were collected and incubated on ice for 20 minutes and centrifuged. Supernatants were kept at −80° C. (intracellular medium). The concentration of nitrite, derived from NO, was measured in the extracellular and the intracellular media. Results are presented in FIG. 6 (A and B).

### Example 7

### NO Production in C6 Alia Cells (Rat Glioma)

All reagents for cellular biology experiments were purchased from Invitrogen. C6 cells from rat glioma were cultured in DMEM, supplemented with 10% fetal bovine serum, streptomycin sulphate (100 μg/ml), and penicillin G (100 U/ml). Cultures were kept at 37° C. in 5% CO₂ atmosphere. 24 hours before peptides were added, cells were transferred to a 24 well plate (1.5×10⁵ cells/ml/well), containing DMEM medium lacking serum. Peptides were added to the cultures to a final concentration of 1 μM, and incubated for 24 hours at 37° C. in 5% CO₂ atmosphere. The medium was collected and stored at −80° C.

NO concentration was determined in 1 ml of culture medium, which was incubated with N-ethylmaleimide at a final concentration of 10 mM, for 24 hours. The supernatant of a centrifugation at 1.000 rpm was injected in the NOA equipment (Nitric Oxide Analyser-Sievers) for NO quantification. Results are presented in FIG. 7.

### Example 8

### Bioavailability of Peptide SEQ ID NO 8 in Mice

Labeling of SEQ ID NO 8 with ¹²⁵I and its Purification

Peptide SEQ ID NO 8 was labeled with ¹²⁵I according to Greenwood and Hunter (Greenwood F C and Hunter W M. The preparation of ¹²⁵I-labelled human growth hormone of high specific radioactivity. J. Biochem., 89:114-123, 1963), with modifications [Biscayart P L, Paladini A C, Vita N, Roguin L P. Preparation of ¹²⁵I-labeled human growth hormone of high quality binding properties endowed with long-term stability. J. Immunoassay, 10, 37-56, 1989; Ribela et al., Protein Exp. Purif., 18(2):115-200, 2000). Under constant shaking, 5 μg of the synthetic SEQ ID NO 8 peptide in PBS were mixed with 1 mCi of ¹²⁵I, and 0.8 μg of chloramines T. After 5 minutes, 1 μg metabisulfite and 200 μg potassium iodine were added to finalize the labeling reaction. Purification of the labeled product was performed by reverse-phase chromatography in a disposable micro-column (Sep-Pak C18, Waters) as described by Christophe et al. [Christophe et al., The synthetic peptide trp-lys-tyr-met-val-met-NH₂ specifically activates neutrophils through FPRL1/lipoxin A4 receptors and is an agonist for the orphan monocyte-expressed chemoattractant receptor FPRL2. J. Biol. Chem. 276: 21585-21593, 2001]. The reaction product was diluted in three volumes of destined water, and applied to the column that had been pre-treated with destined water. Potassium iodine (5 mM, 20 mL) was added, and the column was washed twice with 20 mL distilled water. The labeled peptide was eluted with 2 mL 100% methanol, and 5 μl aliquots were analyzed in the gamma radiation counter from Nuclear Chicago.

### Bioavailability of Peptide SEQ ID NO 8 in Mice

Analysis of the distribution kinetics of the synthetic peptide SEQ ID NO 8 was performed according to Nascimento et al., (Nascimento et al., Effects of gamma radiation on snake venoms. Radiat. Phys. Chem. 52: 665-669, 1998) and Cardi et al., (Cardi B A, Nascimento N, Andrade Jr H F, Irradiation of Crotalus durissus terrificus crotoxin with ⁶⁰Co gamma-rays induces its uptake by macrophage through scavenger receptors. Int. J. Radiat. Biol. 73, 557-564, 1998). For each experiment 80 male albino Swiss mice, weighing approximately 20-25 g, divided into groups of 10 animals, were inoculated into the caudal vein (i.v.) with 50 μL of saline containing a mean of 1×10⁶ cpm of the SEQ ID NO 8/¹²⁵I peptide, plus cold SEQ ID NO 8 peptide, reaching 0.91 mg of peptide/Kg body weight.

The animals were anesthetized after 1, 5, 15, 30, 45, 60, 120 and 180 min after injection, and killed by cervical dislocation, and organs (heart, spleen, liver, kidney, intestines, stomach, thyroid, testicles, and brain) were removed, flushed free of blood with 0.9% saline, and weighed. The radioactivity of each organ was determined in a gamma counter. Tails were collected and submitted to radioactive determination, to be used for dose correction.

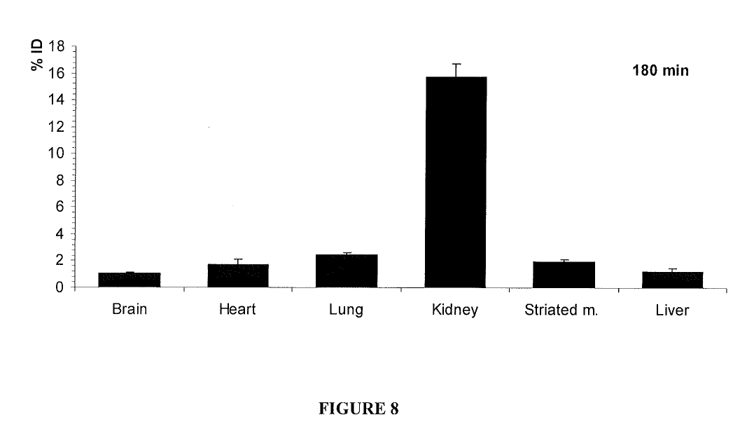

The dose percentage present in each organ was determined, using an administered dose standard. Radioactivity values were registered, given in dose percentage/mg of tissue. Results are presented in FIG. 8.

### Example 9

### Determination of Z-Pro Metabolites in Mice Urine

In vivo hydrolysis of peptides SEQ ID NO 1, SEQ ID NO 4, SEQ ID NO 5, SEQ ID NO 8, and SEQ ID NO 15 was investigated in Swiss male mice weighing approximately 22-25 g, which had free access to food and water, and were kept under a light-dark cycle of 12 hour periods.

Groups of 10 mice were treated intraperitoneally with 0.91 mg/Kg of each proline rich oligopeptide per animal, diluted in 200 μl saline. Treated and control animals (not treated) were kept in metabolic cages, and their urine was collected after 24 hours. The urine was pre-purified in a reverse phase Sep-Pak C18 micro column (Waters), which had been previously equilibrated with solvent A (H₂O/0.1% TFA). Samples were eluted with 60% solvent B (90% acetonitrile/10% solvent A), lyophilized, and resuspended in 1 ml deionized water to be centrifuged at 14.000 rpm for 5 minutes. The supernatant (500 μl) was fractionated in an analytic HPLC system. The gradient was 5-65% solvent B for 30 minutes, using a flow rate of 1 ml/min and monitoring at 214 nm. Fractions were lyophilized and ressuspended in acetonitrile/H₂O (1:1), containing 0.2% formic acid, and were analyzed by mass spectrometry (Q-TOF-Pro). Urine fractions from control and treated animals were compared to determine the presence of metabolites originated from the peptides being tested. Results are presented in Table 2.

TABLE 2

| | Sequence | | Metabolites |
|---|---|---|---|
| SEQ ID NO | and Mr (Da) | | in the urine |
| SEQ ID NO 1 | <EKWAP | 611.7 | <EKW |
| SEQ ID NO 4 | <EDGPIPP | 705.8 | <EDGPIPP |
| SEQ ID NO 5 | <EWPRPQIPP | 1101.3 | <EWPRPQIPP |
| | | | <EWPRPQIP |
| | | | <EWPRP |
| SEQ ID NO 8 | <ENWPHPQIPP | 1196.3 | <ENWPHPQIPP |
| | | | <ENWPHPQIP |
| SEQ ID NO 14 | <EWGRPPGPPIPP | 1281.5 | <EWGRPPGPPIPP |

### Example 10

### Activation of Kidney NOS by Peptides of this Invention

Male Swiss albinos weighing 22-25 g were injected intraperitoneally with: (1) 50 μl 0.9% NaCl (basal level control), or with 0.9% NaCl supplemented with LPS (2 mg/Kg) (positive control); (2) SEQ ID NO 8 (0.21, 0.91, and 3.47 mg/Kg); (3) SEQ ID NO 8 (0.91 mg/Kg) injected into animals pre-treated via intraperitoneally with L-NAME (3 mg/Kg for 1 h); (4) SEQ ID NO 8 (0.91 mg/Kg) injected into animals pre-treated via intraperitoneally with HOE140 (10 μg/Kg for 1 h); (5) captopril (0.21, 0.91, and 3.47 mg/Kg). After 5, 15, 30, 60, 120, and 180 min, animals were killed, their kidneys removed and stored at −70° C. Animals of group 3 and 4 were killed 30 minutes after administration of the oligopeptide.

NOS activity was measured according to Bredt and Snyder [Bredt D S and Snyder S H. Isolation of nitric oxide synthetase, a calmodulin-requiring enzyme. Proc. Natl. Acad. Sci. USA, 87, 682-685, 1990], and is based on estimating the enzymatic activity of the conversion of L-arginine into L-citrulline using ³H-arginine as a reference for the conversion.

NOS Activity Assay

The kidneys were minced in homogenizing buffer, pH 7.4 (20 mM HEPES, 0.32 M saccharose, 1 mM DTT, 0.1 mM EDTA, 1 mM PMSF), and sonicated. Protein dosage was performed according to Bradford (1976), followed by a chromatography step in a 300 μL Dowex 50WX8-400 column.

For the enzymatic assay, 80 μg total protein from each sample were incubated for 60 minutes at 37° C. in 200 μL buffer (30 mM HEPES, 1 mM EDTA, 1.25 mM CaCl₂, 1 mM NADPH, 10 μg/mL calmodulin, 4 μM FAD, 4 μM FMN, 25 μM BH4, 120 nM arginine, 0.5 μCi ³H-arginine). The reaction was stopped by the addition of 1 ml 20 mM HEPES, and incubation in ice bath. ³H-citrulline was separated from ³H-arginine by ion-exchange chromatography in a 300 μL Dowex 50WX8-400 column. Radioactivity of the eluates was measured in a beta-counter. After converting cpm (counts per minute) into dpm (disintegration per minute), the specific activity of the nitric oxide synthase (NOS) was calculated using the following equation:

$$Activity of NOS ( pmol \text{/} min \cdot mg ) = \frac{[ arginine ] \times {}^{3} H \text{-} citrulline ( dpm )}{ \begin{matrix}{}^{3} H \text{-} arginine ( dpm ) \times \\ time ( min ) \times protein ( mg )\end{matrix}}$$

in which ³H-arginine corresponds to the counts of the mixture of cold arginine and ³H-arginine (1:1). The same procedure was used for the negative control of the reaction (blank), except for the addition of the crude extract of kidney tissue. Results are presented in FIG. 9.

### Example 11

### NO Production in Total Kidney Protein Extract, Activated by the Peptides of this Invention

The release of NO was evaluated by means of nitrate and nitrit accumulation in total kidney protein extract of male Swiss mice weighing 30-35 g, as described by Wu and Yen (Wu C C and Yen M H. Higher level of plasma nitric oxide in spontaneously hypertensive rats. Am. J. Hypertension 12: 476-482, 1999). Animals were subjected to perfusion with 0.9% saline solution containing heparin (1:1000). Kidneys were collected and homogenized in cold 50 mM Tris-HCl, 0.1 mM EDTA, 0.1 mM EGTA, 12 mM β-mercaptoethanol, and 1 mM phenylmethylsulphonyl fluoride, pH 7.4 buffer in a homogenizer (Polytron PT MR 3000, Kinematic AG, Littau). Homogenates were incubated with 10 nM, 1 μM, or 10 μM peptide SEQ ID NO 8 in a final volume of 500 μl for 1 hour. Trichloracetic acid (1%) was added at 4° C., and after 20 minutes the mixture was centrifuged at 13.000 g for 7 min at 4° C. A saturated solution of vanadium chloride (VCl₃) in 1 M HCl at 90° C. was added to the supernatant, and the concentration of NO was determined by gas chemiluminescence by means of the reaction of NO with ozone in a NO analyzer (NOA^{TM280}; Sievers Inc.). The concentration of nitrate was determined using a standard curve of NaNO₃ and the Bag software 2.2 (Sievers Instruments Inc.). Results are presented in FIG. 10.

### Example 12

### Anti-Hypertensive Effect of Peptides Described in the Present Invention on Spontaneously Hypertensive Rats (SHR)

In vivo studies on the effect of peptides of the present invention were performed on adult male spontaneously hypertensive rats, and normotensive Wistar rats (Rattus novergicus) weighing 250 to 350 g. Animals had free access to chow (Nuvilab) and water, and were submitted to a light-dark cycle of 12 hours each. Procedures involving animals and their care were conducted according to Giles [Giles A. R. Guidelines for the use of animals in biomedical research. Thromb. Haemost. 58: 1078-1084, 1987].

Polyethylene catheters (PE-10 connected to PE-50) were introduced into abdominal artery through the left femoral artery to measure cardiovascular parameters, and into right femoral vein for intravenous injection after animals had been submitted to tribromoethanol anesthesia (250 mg/Kg body weight). After catheter implant, animals were kept in individual laboratory cages with free access to chow and water for post-surgery recovery during 24 hours.

The cardiovascular parameters (pulse arterial pressure (PAP), mean arterial pressure (MAP), and heart rate (HR)) were monitored by a solid-state strain gauge transducer connected to a computer through a data acquisition system (MP 100; BIOPAC Systems, Inc, Santa Barbara, Calif., USA). The PAP, MAP and HR were continuously presented in different monitor channels and recorded in the computer hard disk for late analysis.

Before drug administration, cardiovascular parameters of the rats were monitored for 40 minutes. Intravenous (I.V.) injection of Ang I (100 ng) was performed in bolus in a total volume of 0.1 mL. 60 minutes after the start of register, 71 nmoles/Kg body weight of peptides SEQ ID NO 8 or SEQ ID NO 4, or captopril were administered I.V. in a total volume of 0.5 mL of 0.9% NaCl solution. Injection of Ang I was repeated 3 to 10 minutes after the administration of peptides SEQ ID NO 8 or SEQ ID NO 4, or captopril, as had been done at the beginning of the experiment. As control, saline was injected instead of peptides SEQ ID NO 8 or SEQ ID NO 4, or captopril. Arterial blood pressure and heart frequency were continuously monitored for 360 minutes.

Results were expressed as the mean variation of MAP±standard deviation. For statistical analysis of the experiments the One-Way ANOVA software was used, followed by the Bonferroni test performed with the GraphPad Prism 4.0 (GraphPad Software, Inc.). Results are presented in FIGS. 11 A-E, 12, and 13.

### Example 13

### Anti-Hypertensive Action of Peptides SEQ ID NO 8, SEQ ID NO 1, SEQ ID NO 4 and Captopril on SHR Anaesthetized with Pentobarbitone

Adult male SHR, weighing between 250 and 350 g, were used. Material and methods described in Example 12 were employed, except for those described below.

Two different experiment sets were performed:

1) Five SHR per group were anaesthetized intraperitoneally with 60 mg/Kg body weight with sodium pentobarbitone. Mean arterial blood pressure and heart rate were continuously calculated from arterial blood pressure pulses (PAP). Cardiovascular parameters were monitored during 30 minutes before i.v. administration of peptides SEQ ID NO 1, or SEQ ID NO 4, or SEQ ID NO 8 (71 nmoles/Kg body weight), or captopril (10 μmoles/Kg body weight), in a final volume of 0.5 mL 0.9% NaCl solution. The control was the vehicle (0.9% NaCl). PAM and heart rate were continuously monitored up to 180 minutes after administration of the anti-hypertensive compound. Results are presented in FIG. 14 A.

2) The day before measurements, the SHR underwent surgery for catheter implantation into the femoral artery and vein. Anesthesia was performed with sodium pentobarbital at 60 mg/Kg of body weight. After catheter implantation the animals were kept in individual laboratory cages having free access to food and water for 24 hours for post-surgery recovery. Cardiovascular parameters of the rats were monitored for 60 minutes before i.v. administration of peptides SEQ ID NO 1, or SEQ ID NO 4, or SEQ ID NO 8 (71 nmoles/Kg body weight), or captopril (10 μmoles/Kg body weight), in a final volume of 0.5 mL 0.9% NaCl solution. Control was the vehicle (0.9% NaCl). MAP and heart rate were continuously monitored up to 360 minutes after administration of the peptides.

Results, presented in FIG. 14B, are expressed as mean±SEM. Comparisons were made by Student unpaired t test or one-way ANOVA with Dunnett post-test, when appropriate (GraphPad Prism 4.0, GraphPad Software, Incorporation).

### Example 14

### Effect of High Doses of Peptides of the Present Invention on Normotensive Rats

Adult male Wistar normotensive rats, weighing between 250 and 350 g, were used in the experimental evaluation of the hypotensive effect of SEQ ID NO 8, as described in Example 12. An i.v. dose of 10 μmoles/Kg of body weight of peptide SEQ ID NO 8 was injected, and arterial blood pressure was monitored for 6 hours (results not shown).

Results showed that peptide SEQ ID NO 8, at the dose of 10 μmoles/Kg of body weight, had no significant hypotensive effect during the 6 hours the arterial blood pressure of these animals was being measured (results not shown).

1.-25. (canceled)

26. An oligopeptide comprising an amino acid sequence selected from the group consisting of SEQ ID NOS: 2, 6-11, 17 and 18, having at least one activity of substantially enhancing and sustaining nitric oxide production in animal cells, potentiating argininosuccinate synthase activity of animal cells, and increasing the intracellular concentration of bivalent calcium ion in animal cells.

27. The oligopeptide of claim 26 having the activity of potentiating argininosuccinate synthase activity of endothelial and nerve cells.

28. The oligopeptide of claim 26 having the activity of increasing the intracellular concentration of bivalent calcium ion in endothelial and nerve cells.

29. The oligopeptide of claim 26, having the activity of potentiating the nitric oxide synthase activity in renal cells.

30. The oligopeptide of claim 26 having the activity of activating and sustaining nitric oxide production in renal cells.

31. The oligopeptide of claim 26, wherein said oligopeptide concentrates in renal tissue for a period longer than 3 hours.

32. The oligopeptide of claim 26 having a chemical modification at the amino terminus, or at the carboxy terminus, or at both ends.

33. An oligopeptide consisting of an amino acid sequence selected from the group consisting of SEQ ID NOS: 2, 6-11, 17 and 18.

34. The oligopeptide of claim 26 that specifically binds to an argininosuccinate synthase of animal cells.

35. A pharmaceutical composition comprising from 0.05 μg to 10 mg of at least one oligopeptide of claim 26, or a mixture thereof, or pharmaceutically acceptable salts or solvates thereof, and one or more pharmaceutically acceptable additives.

36. The pharmaceutical composition of claim 35, comprising from 0.5 μg to 5 mg of the at least one oligopeptide, or mixture thereof.

37. The pharmaceutical composition of claim 35, comprising 0.1 μg to 0.01 mg of the at least one oligopeptide, or mixture thereof.

38. The pharmaceutical composition of claim 35, having activity of reducing the arterial blood pressure in hypertensive mammals for a period of at least 3 hours after administration to said mammal.

39. A method of activating nitric oxide biosynthesis, and/or potentiating argininosuccinate synthase activity, and/or increasing intracellular concentration of bivalent calcium ion, in a mammal comprising administering to said mammal an oliopeptide comprising an amino acid sequence selected from the group consisting of the amino acid sequences set forth in SEQ ID NOS: 2, 6-11, 17 and 18, having at least one activity of substantially enhancing and sustaining nitric oxide production in animal cells, potentiating the argininosuccinate synthase activity of animal cells, and increasing the intracellular concentration of bivalent calcium ion in animal cells.

40. A method for treatment of one or more disorders in a mammal selected from the group consisting of cardiovascular diseases, disorders of the nervous system, disorders of the gastrointestinal system, disorders of the immune system, disorders of systemic hemodynamics, renal hemodynamics, coronary hemodynamics, neurodegenerative pathologies, pre-eclampsia, lymphocyte dysfunction in the immune response, tumor growth, erectile dysfunction, and control of production of germinative cells in a mammal, comprising administering to the mammal an oligopeptide comprising an amino acid sequence selected from the group consisting of the amino acid sequences set forth in SEQ ID NOS: 2, 6-11, 17 and 18, having at least one activity of substantially enhancing and sustaining nitric oxide production in animal cells, potentiating the argininosuccinate synthase activity of animal cells, and increasing the intracellular concentration of bivalent calcium ion in animal cells.

41. The method of claim 40, in which the oligopeptide is administered in association with one or more medicaments that improves the therapeutic effects of the oligopeptide.

42. A method of treating a disorder in a mammal caused by nitric oxide deficiency comprising administering the pharmaceutical composition comprising from 0.05 μg to 10 mg of at least one oligopeptide selected from the group consisting of SEQ ID NOS: 2, 6-11, 17 and 18, or a mixture thereof, or pharmaceutically acceptable salts or solvates thereof, and one or more pharmaceutically acceptable additives, to said mammal.

43. A method for lowering blood pressure in a mammal comprising administering the pharmaceutical composition comprising from 0.05 μg to 10 mg of at least one oligopeptide selected from the group consisting of SEQ ID NOS: 2, 6-11, 17 and 18, or a mixture thereof, or pharmaceutically acceptable salts or solvates thereof, and one or more pharmaceutically acceptable additives, to said mammal.
